(19) United States
(12) Patent Application Publication
(10) Pub. No.: US 2007/0135431 A1
(43) Pub. Date: Jun. 14, 2007

(54) INHIBITORS OF HISTONE DEACETYLASE FOR THE TREATMENT OF DISEASE

(75) Inventors: Nicholas D. Smith, San Diego, CA (US); Celine Bonnefous, San Diego, CA (US); Joseph E. Payne, San Diego, CA (US); Timothy Z. Hoffman, San Diego, CA (US); Paul L. Wash, San Diego, CA (US); Christian A. Hassig, San Diego, CA (US); Shawn A. Scranton, San Diego, CA (US)

Correspondence Address: INTERNATIONAL PATENT GROUP;ATTN: MS LAVERN HALL, P.O. BOX 38129, ST. LOUIS, MO, 63138, US

(73) Assignee: KALYPSYS, INC, San Diego, CA (US)

(21) Appl. No.: 11/608,726

(22) Filed: Dec. 8, 2006

Related U.S. Application Data
(60) Provisional application No. 60/748,823, filed on Dec. 9, 2005.
(60) Provisional application No. 60/802,823, filed on May 22, 2006.

(51) Int. Cl. A61K 31/5377; A61K 31/454; A61K 31/4439; C07D 413/14; C07D 401/14

(52) U.S. Cl. 514/235.2; 514/326; 514/340; 544/124; 546/207; 546/277.1

(57) ABSTRACT

Disclosed herein are carbonyl compounds of having the structural formula: 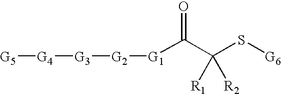 or a salt, ester, or prodrug thereof, Methods and compositions are disclosed for treating disease states including, but not limited to cancers, autoimmune diseases, tissue damage, central nervous system disorders, neurodegenerative disorders, fibrosis, bone disorders, polyglutamine-repeat disorders, anemias, thalassemias, inflammatory conditions, cardiovascular conditions, and disorders in which angiogenesis play a role in pathogenesis, using the compounds of the invention. In addition, methods of modulating the activity of histone deacetylase (HDAC) are also disclosed.

This application claims the benefit of priority of U.S. provisional application No. 60/748,823, filed Dec. 9, 2005, and U.S. provisional application No. 60/802,823, filed May 22, 2006, the disclosures of which are hereby incorporated by reference as if written herein in their entireties.

### FIELD OF THE INVENTION

The present invention is directed to carbonyl compounds as inhibitors of histone deacetylase (HDAC). These compounds are useful in treating disease states including cancers, autoimmune diseases, tissue damage, central nervous system disorders, neurodegenerative disorders, fibrosis, bone disorders, polyglutamine-repeat disorders, anemias, thalassemias, inflammatory conditions, cardiovascular conditions, and disorders in which angiogenesis plays a role in pathogenesis.

### BACKGROUND OF THE INVENTION

Histone proteins organize DNA into nucleosomes, which are regular repeating structures of chromatin. The acetylation status of histones alters chromatin structure, which, in turn, is involved in gene expression. Two classes of enzymes can affect the acetylation of histones—histone acetyltransferases (HATs) and histone deacetylases (HDACs). A number of HDAC inhibitors have been characterized. One of the potent inhibitors of HDAC is (SAHA), a hydroxamic acid-based compound. It is also known as vorinostat or ZOLINZA(TM), which is currently in clinical trials. (“Merck Announces Pivotal Phase IIb Study Results of the Company's Investigational HDAC Inhibitor ZOLINZA(TM) and Glaxo's Cancer Vaccine Shows Response,” M2 Presswire, 5 Jun. 2006.) The Food and Drug Administration (FDA) has also accepted the New Drug Application (NDA) for ZOLINZA(TM) for the treatment of advanced cutaneous T-cell-lymphoma (CTCL) in Jun. 2006. (WHITEHOUSE STATION, N.J., “ZOLINZA(TM), Merck's Investigational Medicine for Advanced Cutaneous T-Cell Lymphoma (CTCL), to Receive Priority Review from U.S. Food and Drug Administration,” Business Wire, 7 Jun. 2006.)

Certain non-hydroxamate carbonyl compounds as HDAC inhibitors have previously been published. In PCT Patent Application Publication No. WO 04/110418, published Dec. 23, 2004, Wash et al. first discloses a novel, non-hydroxamate carbonyl class of HDAC inhibitor. In PCT Patent Application Publication No. WO 05/123089, published Dec. 29, 2005, Malecha et al. describes multicyclic sulfonamide carbonyl compounds as HDAC inhibitors. In PCT Patent Application Publication No. WO 05/120515, published Dec. 29, 2005, Malecha et al. discloses somewhat different sulfonamide carbonyl compounds as HDAC inhibitors.

### SUMMARY OF THE INVENTION

Disclosed herein are carbonyl compounds, including their salts, esters, and prodrugs, having structural Formula (I) or related formulae as described herein:

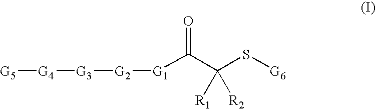

wherein:

G₁ is selected from the group consisting of optionally substituted 5 or 6 membered aryl and optionally substituted 5 or 6 membered heteroaryl;

G₂ is selected from the group consisting of an N-sulfonamide moiety having structure (II), an S-sulfonamide moiety having structure (III), an amide of the form —NR₃C(O)—, and an amide of the form —C(O)NR₃—:

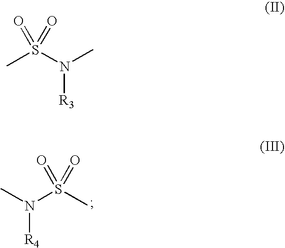

G₃ is selected from the group consisting of optionally substituted phenyl, optionally substituted 5 or 6 membered aryl, and optionally substituted 5 or 6 membered heteroaryl;

R₁ and R₂ are each independently selected from the group consisting of hydrogen, lower alkyl, halogen and perhaloalkyl, or R₁ and R₂ taken together may form an optionally substituted cycloalkyl or optionally substituted heterocycloalkyl;

R₃ and R₄ are each independently selected from the group consisting of hydrogen, optionally substituted lower alkyl, and optionally substituted aryl;

G₄ is selected from the group consisting of —(CR₅R₆)ₘ—, —(X₁)ₙ₁O(X₂)ₙ₂—, —(X₁)ₙ₁NR₇(X₂)ₙ₂—, —SO₂—, —(X₁)ₙ₁C(O)NR₇(X₂)ₙ₂—, and —(X₁)ₙ₁NR₇C(O)(X₂)ₙ₂—, wherein each may be optionally substituted with one or more R₉ attached to any carbon atom;

R₅ and R₆ are each independently selected from the group consisting of hydrogen, optionally substituted lower alkyl, optionally substituted lower alkoxy, optionally substituted aryl, and optionally substituted lower perhaloalkyl;

R₇ is selected from the group consisting of hydrogen, optionally substituted lower alkyl, optionally substituted heteroalkyl, and optionally substituted lower alkoxy;

R₉ is selected from the group consisting of lower alkyl, lower alkylene, lower alkynylene, lower alkoxy, lower amino, halogen, lower perhaloalkyl, and hydroxyl;

m is 1-6;

X₁ and X₂ are each independently optionally substituted lower alkylene, optionally substituted alkenylene, and optionally substituted alkynylene;

n1 is 0-5;

n2 is 0-5;

G₅ is selected from the group consisting of optionally substituted aryl, optionally substituted heteroaryl, optionally substituted cycloalkyl, optionally substituted heterocycloalkyl, optionally substituted fused aryl, optionally substituted fused heteroaryl, optionally substituted fused heterocycloalkyl, and optionally substituted fused cycloalkyl; and

G₆ is selected from the group consisting of hydrogen, optionally substituted acyl, optionally substituted aryl, optionally substituted alkyl, optionally substituted heteroaryl, optionally substituted alkylthio, optionally substituted arylthio and optionally substituted heteroarylthio; or G₆ may have the structural formula (IV) thereby forming a homodisulfide or heterodisulfide dimer of a compound of the present invention

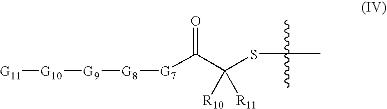

wherein:

G₇ is selected from the group consisting of optionally substituted 5 or 6 membered aryl and optionally substituted 5 or 6 membered heteroaryl;

G₈ is selected from the group consisting of an N-sulfonamide moiety having structure (V), an S-sulfonamide moiety having structure (VI), an amide of the form —NR₁₂C(O)—, and an amide of the form —C(O)NR₁₂—:

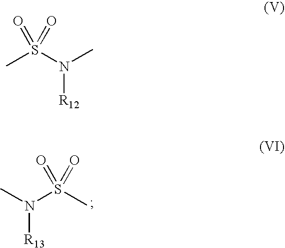

G₉ is selected from the group consisting of optionally substituted phenyl, optionally substituted 5 or 6 membered aryl, and optionally substituted 5 or 6 membered heteroaryl;

R₁₀ and R₁₁ are each independently selected from the group consisting of hydrogen, lower alkyl, halogen and perhaloalkyl, or R₁₀ and R₁₁ taken together may form an optionally substituted cycloalkyl or optionally substituted heterocycloalkyl;

R₁₂ and R₁₃ are each independently selected from the group consisting of hydrogen, optionally substituted lower alkyl, and optionally substituted aryl;

G₁₀ is selected from the group consisting of —(CR₁₄R₁₅)_{q}—, —(X₃)ᵣ₁O(X₄)ᵣ₂—, —(X₃)ᵣ₁NR₁₆(X₄)ᵣ₂—, —SO₂—, —(X₃)ᵣ₁C(O)NR₁₆(X₄)ᵣ₂—, and —(X₃)ᵣ₁NR₁₆C(O)(X₄)ᵣ₂—, wherein each may be optionally substituted with one or more R₁₇ attached to any carbon atom;

R₁₄ and R₁₅ are each independently selected from the group consisting of hydrogen, optionally substituted lower alkyl, optionally substituted lower alkoxy, optionally substituted aryl, and optionally substituted lower perhaloalkyl;

R₁₆ is selected from the group consisting of hydrogen, optionally substituted lower alkyl, optionally substituted heteroalkyl, and optionally substituted lower alkoxy;

R₁₇ is selected from the group consisting of lower alkyl, lower alkylene, lower alkynylene, lower alkoxy, lower amines, halogen, lower perhaloalkyl, and hydroxyl;

q is 1-6;

X₃ and X₄ are each independently optionally substituted lower alkylene, optionally substituted alkenylene, and optionally substituted alkynylene;

r1 is 0-5;

r2 is 0-5; and

G₁₁ is selected from the group consisting of optionally substituted aryl, optionally substituted heteroaryl, optionally substituted cycloalkyl, optionally substituted heterocycloalkyl, optionally substituted cycloalkenyl, optionally substituted fused aryl, optionally substituted fused heteroaryl, optionally substituted fused heterocycloalkyl, and optionally substituted fused cycloalkyl.

In a broad aspect, compounds according to the present invention are capable of inhibiting the catalytic activity of histone deacetylase (HDAC), and may be used in the treatment or prophylaxis of a disease or condition in which HDAC plays an active role. Thus, in broad aspect, the present invention provides methods and pharmaceutical compositions comprising one or more compounds of the present invention together with a pharmaceutically acceptable carrier, for treating diseases in mammals using compounds of the invention, including but not limited to, treating cancers, autoimmune diseases, tissue damage, central nervous system disorders, neurodegenerative disorders, fibrosis, bone disorders, polyglutamine-repeat disorders, anemias, thalassemias, inflammatory conditions, cardiovascular conditions, and disorders in which angiogenesis plays a role in pathogenesis.

In certain embodiments, the present invention provides methods for inhibiting the catalytic activity and cellular function of HDAC. In other embodiments, the present invention provides methods for treating a HDAC mediated disorder in a patient in need of such treatment comprising administering to said patient a therapeutically effective amount of a compound or composition according to the present invention. The present invention also contemplates the use of compounds disclosed herein for use in the manufacture of a medicament for the treatment of a disease or condition ameliorated by the inhibition/modulation of HDAC.

### DETAILED DESCRIPTION OF THE INVENTION

In certain embodiments, G₂ is selected from the group consisting of S-sulfonamide and N-sulfonamide.

In certain embodiments, G₆ is selected from the group consisting of optionally substituted acyl and hydrogen.

In certain embodiments, G₂ is N-sulfonamide.

In certain embodiments, G₃ is phenyl.

In certain embodiments, G₄ is —(X₁)ₙ₁O(X₂)ₙ₂— and n1 is 0.

In certain embodiments, G₅ is selected from the group consisting of optionally substituted heterocycloalkyl, optionally substituted heteroaryl or optionally substituted aryl.

In further embodiments, G₅ is optionally substituted heterocycloalkyl.

In other embodiments, G₁ is pyridinyl.

In other embodiments, G₁ is phenyl.

In other embodiments, G₄ is —(CR₅R₆)ₘ—.

In yet other embodiments, G₄ is —(X₁)ₙ₁NR₇(X₂)_{n2— and n}1 is 0.

In certain embodiments, G₅ is selected from the group consisting of optionally substituted heterocycloalkyl, optionally substituted heteroaryl or optionally substituted aryl.

In accordance with one aspect, the present invention provides compounds capable of inhibiting the catalytic activity of histone deacetylase (HDAC). In another aspect, the present invention provides pharmaceutical compositions comprising compounds capable of inhibiting the catalytic activity of histone deacetylase (HDAC).

In accordance with yet another aspect of the invention, the present invention provides methods and compositions for treating certain diseases or disease states.

In another aspect are compounds or compositions comprising compounds capable of inhibiting the catalytic activity of histone deacetylase (HDAC).

In some aspects of the invention, the disease is a hyperproliferative condition of the human or animal body.

In another aspects of the invention, said hyperproliferative condition is selected from the group consisting of hematologic and nonhematologic cancers. In yet further embodiments, said hematologic cancer is selected from the group consisting of multiple myeloma, leukemias, and lymphomas. In yet another aspects of the invention, said leukemia is selected from the group consisting of acute and chronic leukemias. In yet further embodiments, said acute leukemia is selected from the group consisting of acute lymphocytic leukemia (ALL) and acute nonlymphocytic leukemia (ANLL). In yet further embodiments, said chronic leukemia is selected from the group consisting of chronic lymphocytic leukemia (CLL) and chronic myelogenous leukemia (CML). In further embodiments, said lymphoma is selected from the group consisting of Hodgkin's lymphoma and non-Hodgkin's lymphoma. In further embodiments, said hematologic cancer is multiple myeloma. In other embodiments, said hematologic cancer is of low, intermediate, or high grade. In other embodiments, said nonhematologic cancer is selected from the group consisting of: brain cancer, cancers of the head and neck, lung cancer, breast cancer, cancers of the reproductive system, cancers of the digestive system, pancreatic cancer, and cancers of the urinary system. In further embodiments, said cancer of the digestive system is a cancer of the upper digestive tract or colorectal cancer. In further embodiments, said cancer of the urinary system is bladder cancer or renal cell carcinoma. In further embodiments, said cancer of the reproductive system is prostate cancer.

Additional types of cancers which may be treated using the compounds and methods described herein include: cancers of oral cavity and pharynx, cancers of the respiratory system, cancers of bones and joints, cancers of soft tissue, skin cancers, cancers of the genital system, cancers of the eye and orbit, cancers of the nervous system, cancers of the lymphatic system, and cancers of the endocrine system. In certain embodiments, these cancer s may beselected from the group consisting of: cancer of the tongue, mouth, pharynx, or other oral cavity; esophageal cancer, stomach cancer, or cancer of the small intestine; colon cancer or rectal, anal, or anorectal cancer; cancer of the liver, intrahepatic bile duct, gallbladder, pancreas, or other biliary or digestive organs; laryngeal, bronchial, and other cancers of the respiratory organs; heart cancer, melanoma, basal cell carcinoma, squamous cell carcinoma, other non-epithelial skin cancer; uterine or cervical cancer; uterine corpus cancer; ovarian, vulvar, vaginal, or other female genital cancer; prostate, testicular, penile or other male genital cancer; urinary bladder cancer; cancer of the kidney; renal, pelvic, or urethral cancer or other cancer of the genito-urinary organs; thyroid cancer or other endocrine cancer; chronic lymphocytic leukemia; and cutaneous T-cell lymphoma, both granulocytic and monocytic.

Yet other types of cancers which may be treated using the compounds and methods described herein include: adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, hemangioblastoma, hepatocellular carcinoma, hepatoma, Kaposi's sarcoma, large cell carcinoma, leiomyosarcoma, liposarcoma, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, squamous cell carcinoma, sweat gland carcinoma, synovioma, thyroid cancer, uveal melanoma, and Wilm's tumor.

In some aspects of the invention, the disease to be treated by the methods of the present invention may be a hematologic disorder. In certain embodiments, said hematologic disorder is selected from the group consisting of sickle cell anemia, myelodysplastic disorders (MDS), and myeloproliferative disorders. In further embodiments, said myeloproliferative disorder is selected from the group consisting of polycythemia vera, myelofibrosis and essential thrombocythemia.

In some aspects of the invention, the disease to be treated by the methods of the present invention may be a neurological disorder. In further embodiments, said neurological disorder is selected from the group consisting of epilepsy, neuropathic pain, depression and bipolar disorders.

In some aspects of the invention, the disease to be treated by the methods of the present invention may be a cardiovascular condition. In certain embodiments, said cardiovascular condition is selected from the group consisting of cardiac hypertrophy, idiopathic cardiomyopathies, and heart failure.

In some aspects of the invention, the disease to be treated by the methods of the present invention may be an autoimmune disease. In certain embodiments, said autoimmune disease is selected from the group consisting of systemic lupus erythromatosus (SLE), multiple sclerosis (MS), and systemic lupus nephritis.

In some aspects of the invention, the disease to be treated by the methods of the present invention may be a dermatologic disorder. In certain embodiments, said dermatologic disorder is selected from the group consisting of psoriasis, melanoma, basal cell carcinoma, squamous cell carcinoma, and other non-epithelial skin cancers.

In some aspects of the invention, the disease to be treated by the methods of the present invention may be an ophthalmologic disorder. In certain embodiments, said ophthalmologic disorder is selected from the group consisting of dry eye, closed angle glaucoma and wide angle glaucoma.

In some aspects of the invention, the disease to be treated by the methods of the present invention may be a polyglutamine-repeat disorders. In some embodiments, the polyglutamine-repeat disorder is selected from the group consisting of Huntington's disease, Spinocerebellar ataxia 1 (SCA 1), Machado-Joseph disease (MJD)/Spinocerebella ataxia 3 (SCA 3), Kennedy disease/Spinal and bulbar muscular atrophy (SBMA) and Dentatorubral pallidolusyian atrophy (DRPLA).

In some aspects of the invention, the disease to be treated by the methods of the present invention may be an inflammatory condition. In some embodiments, the inflammatory condition is selected from the group consisting of Rheumatoid Arthritis (RA), Inflammatory Bowel Disease (IBD), ulcerative colitis and psoriasis.

In another aspect are compounds or compositions comprising compounds capable of inhibiting the catalytic or cellular activity of histone deacetylase (HDAC).

As used in the present specification, the following terms have the meanings indicated.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydonapthalene, octahydronapthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions.

Examples Include Fluoromethylene

(—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to 3 to 7 membered, preferably 5 to 7 membered, unsaturated heteromonocyclic rings, or fused polycyclic rings in which at least one of the fused rings is unsaturated, wherein at least one atom is selected from the group consisting of O, S, and N. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of this invention.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —SO₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-menbered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particuar moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The terms “therapy” or “treating” as used herein refer to (1) reducing the rate of progress of a disease, or, in case of cancer reducing the size of the tumor; (2) inhibiting to some extent further progress of the disease, which in case of cancer may mean slowing to some extent, or preferably stopping, tumor metastasis or tumor growth; and/or, (3) relieving to some extent (or, preferably, eliminating) one or more symptoms associated with the disease. Thus, the term “therapeutically effective amount” as used herein refers to that amount of the compound being administered which will provide therapy or affect treatment.

In some aspects of the invention, the compounds of the present invention are also anti-tumor compounds and/or inhibit the growth of a tumor, i.e., they are tumor-growth-inhibiting compounds. The terms “anti-tumor” and “tumor-growth-inhibiting,” when modifying the term “compound,” and the terms “inhibiting” and “reducing”, when modifying the terms “compound” and/or “tumor,” mean that the presence of the subject compound is correlated with at least the slowing of the rate of growth of the tumor. More preferably, the terms “anti-tumor,” “tumor-growth-inhibiting,” “inhibiting,” and “reducing” refer to a correlation between the presence of the subject compound and at least the temporary cessation of tumor growth. The terms “anti-tumor,” “tumor-growth-inhibiting,” “inhibiting,” and “reducing” also refer to, a correlation between the presence of the subject compound and at least the temporary reduction in the mass of the tumor.

The term “function” refers to the cellular role of HDAC. The term “catalytic activity”, in the context of the invention, defines the rate at which HDAC deacetylates a substrate. Catalytic activity can be measured, for example, by determining the amount of a substrate converted to a product as a function of time. Deacetylation of a substrate occurs at the active-site of HDAC. The active-site is normally a cavity in which the substrate binds to HDAC and is deacetylated.

The term “substrate” as used herein refers to a molecule deacetylated by HDAC. The substrate is preferably a peptide and more preferably a protein. In some embodiments, the protein is a histone, whereas in other embodiments, the protein is not a histone.

The term “inhibit” refers to decreasing the cellular function of HDAC. It is understood that compounds of the present invention may inhibit the cellular function of HDAC by various direct or indirect mechanisms, in particular by direct or indirect inhibition of the catalytic activity of HDAC. The term “activates” refers to increasing the cellular function of HDAC.

The term “activate” refers to increasing the cellular function of HDAC.

HDAC function is preferably the interaction with a natural binding partner and most preferably catalytic activity.

The term “modulate” refers to altering the function of HDAC by increasing or decreasing the probability that a complex forms between HDAC and a natural binding partner. A modulator may increase the probability that such a complex forms between HDAC and the natural binding partner, or may increase or decrease the probability that a complex forms between HDAC and the natural binding partner depending on the concentration of the compound exposed to HDAC, or may decrease the probability that a complex forms between HDAC and the natural binding partner. A modulator may activate the catalytic activity of HDAC, or may activate or inhibit the catalytic activity of HDAC depending on the concentration of the compound exposed to HDAC, or may inhibit the catalytic activity of HDAC.

The term “complex” refers to an assembly of at least two molecules bound to one another. The term “natural binding partner” refers to polypeptides that bind to HDAC in cells. A change in the interaction between HDAC and a natural binding partner can manifest itself as an increased or decreased probability that the interaction forms, or an increased or decreased concentration of HDAC/natural binding partner complex.

The term “contacting” as used herein refers to mixing a solution comprising a compound of the invention with a liquid medium bathing the cells of the methods. The solution comprising the compound may also comprise another component, such as dimethylsulfoxide (DMSO), which facilitates the uptake of the compound or compounds into the cells of the methods. The solution comprising the compound of the invention may be added to the medium bathing the cells by utilizing a delivery apparatus, such as a pipet-based device or syringe-based device.

The term “monitoring” refers to observing the effect of adding the compound to the cells of the method. The effect can be manifested in a change in cell phenotype, cell proliferation, HDAC catalytic activity, substrate protein acetylation levels, gene expression changes, or in the interaction between HDAC and a natural binding partner.

The term “effect” describes a change or an absence of a change in cell phenotype or cell proliferation. “Effect” can also describe a change or an absence of a change in the catalytic activity of HDAC. “Effect” can also describe a change or an absence of a change in an interaction between HDAC and a natural binding partner.

The term “cell phenotype” refers to the outward appearance of a cell or tissue or the function of the cell or tissue. Examples of cell phenotype are cell size (reduction or enlargement), cell proliferation (increased or decreased numbers of cells), cell differentiation (a change or absence of a change in cell shape), cell survival, apoptosis (cell death), or the utilization of a metabolic nutrient (e.g., glucose uptake). Changes or the absence of changes in cell phenotype are readily measured by techniques known in the art.

“HDAC inhibitor” is used herein to refer to a compound that exhibits an IC₅₀ with respect to HDAC activity of no more than about 100 μM and more typically not more than about 50 μM, as measured in the biochemical in vitro HDAC-inhibition assay, cellular histone hyperacetylation assay, and differential cytotoxicity assay described generally herein below. “IC₅₀” is that concentration of inhibitor which reduces the activity of an enzyme (e.g., HDAC) to half-maximal level. Representative compounds of the present invention have been discovered to exhibit inhibitory activity against HDAC. Compounds of the present invention preferably exhibit an IC₅₀ with respect to HDAC of no more than about 10 μM, more preferably, no more than about 5 μM, even more preferably not more than about 1 μM, and most preferably, not more than about 200 nM, as measured in the HDAC assays described herein.

The term “prodrug” refers to a compound that is made more active in vivo. Certain compounds of the present invention may also exist as prodrugs, as described in Hydrolysis in Drug and Prodrug Metabolism Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. Yet another example of a prodrug is protected thiol compounds. Thiols bearing hydrolyzable protecting groups can unmask protected SH groups prior to or simultaneous to use. As shown below, the moiety —C(O)—R_{E} of a thioester may be hydrolyzed to yield a thiol and a pharmaceutically acceptable acid HO—C(O)—R_{E}.

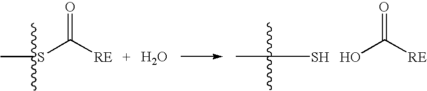

A “pharmaceutically active metabolite” is intended to mean a pharmacologically active product produced through metabolism in the body of a specified compound or salt thereof. Metabolites of a compound may be identified using routine techniques known in the art and their activities determined using tests such as those decribed herein.

The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

The term “thiol protecting group” refers to thiols bearing hydrolyzable protecting groups that can unmask protected SH groups prior to or simultaneous to use. Preferred thiol protecting groups include but are not limited to thiol esters which release pharmaceutically acceptable acids along with an active thiol moiety. Such pharmaceutically acceptable acids are generally nontoxic and do not abrogate the biological activity of the active thiol moiety. Examples of pharmaceutically acceptable acids include, but are not limited to: N,N-diethylglycine; 4-ethylpiperazinoacetic acid; ethyl 2-methoxy-2-phenylacetic acid; N,N-dimethylglycine; (nitrophenoxysulfonyl)benzoic acid; acetic acid; maleic acid; fumaric acid; benzoic acid; tartraric acid; natural amino acids (like glutamate, aspartate, cyclic amino acids such proline); D-amino acids; butyric acid; fatty acids like palmitic acid, stearic acid, oleate; pipecolic acid; phosphonic acid; phosphoric acid; pivalate (trimethylacetic acid); succinic acid; cinnamic acid; anthranilic acid; salicylic acid; lactic acid; and pyruvic acids.

Another aspect of the present invention are compounds containing at least one thiol in a protected form, which can be released to provide a SH group prior to or simultaneous to use. Thiol moieties are known to be unstable in the presence of air and are oxidized to the corresponding disulfide. Protected thiol groups are those that can be converted under mild conditions into free thiol groups without other undesired side reactions taking place. Suitable thiol protecting groups include but are not limited to trityl (Trt), allyloxycarbonyl (Alloc), 1-(4,4-dimethyl-2,6-dioxocyclohex-1-ylidene)ethyl (Dde), acetamidomethyl (Acm), t-butyl (tBu), or the like. Preferred thiol protecting groups include lower alkanoyl, e.g. acetyl. Free thiol, disulfides, and protected thiols are understood to be within the scope of this invention.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “therapeutically acceptable salt,” as used herein, represents salts or zwitterionic forms of the compounds of the present invention which are water or oil-soluble or dispersible; which are suitable for treatment of diseases without undue toxicity, irritation, and allergic-response; which are commensurate with a reasonable benefit/risk ratio; and which are effective for their intended use. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

The compounds of the present invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question.

While it may be possible for the compounds of the subject invention to be administered as the raw chemical, it is also possible to present them as a pharmaceutical formulation. Accordingly, the subject invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

For administration by inhalation the compounds according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

In certain embodiments, pharmaceutical preparations of compound(s) or active ingredient(s) of the present invention may be formulated by Latitude Pharmaceuticals Inc. located in 9865 Mesa Rim Road, STE 201, San Diego, Calif. 92121 using their trade secret and proprietary formulation named “F101”. The composition of said formulation F101 is known to contain triglyceride, soy lecithin, vitamin E and PEG400.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

Further, the compounds of the invention may be administered on a daily basis or on a schedule containing days where dosing does not take place. In certain embodiments, dosing may take place every other day. In other embodiments, dosing may take place for five consecutive days of a week, then be followed by two non-dosing days. The choice of dosing schedule will depend on many factors, including, for example, the formulation chosen, route of administration, and concurrent pharmacotherapies, and may vary on a patient-to-patient basis. It is considered within the capacity of one skilled in the art to select a schedule that will maximize the therapeutic benefit and minimize any potential side effects in a patient.

The amount of active ingredient that may be combined with the carrier materials to produce a single dosage form will vary depending upon the host treated and the particular mode of administration.

The compounds of the subject invention can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for cancer involving administration of one of the compounds described herein, increased therapeutic benefit may result by also providing the patient with another therapeutic agent for cancer. In any case, regardless of the disease, disorder, or condition being treated, the overall benefit experienced by the patient may simply be additive of the two therapeutic agents or the patient may experience a synergistic benefit.

Specific, non-limiting examples of possible combination therapies include use of the compounds of the invention with another chemotherapeutic agent such as aromatase inhibitors, antiestrogen, anti-androgen, or a gonadorelin agonists, topoisomerase land 2 inhibitors, microtubule active agents, alkylating agents, antimeoplastic antimetabolite, or platin containing compound, lipid or protein kinase targeting agents, protein or lipid phosphatase targeting agents, anti-angiogentic agents, agents that induce cell differentiation, bradykinin 1 receptor and angiotensin II antagonists, cyclooxygenase inhibitors, heparanase inhibitors, lymphokines or cytokine inhibitors, bisphosphanates, rapamycin derivatives, anti-apoptotic pathway inhibitors, apoptotic pathway agonists, PPAR agonists, inhibitors of Ras isoforms, telomerase inhibitors, protease inhibitors, metalloproteinase inhibitors, aminopeptidase inhibitors, and biologic drugs including but not limited to antibodies, cytokines and growth factors.

In some aspects of the invention, the chemotherapeutic agents that are useful for the treatment of multiple myeloma include, but are not limited to, alkylating agents (eg, melphalan), anthracyclines (eg. doxorubicin), corticosteroids (eg. dexamethasome), IMiDs (eg. thalidomide, lenalidomide), protease inhibitors (eg. bortezomib, NPI0052), IGF-1 inhibitors, CD40 antibodies, Smac mimetics (eg. telomestatin), FGF3 modulator (eg. CHIR258), mTOR inhibitor (Rad 001), HDAC inhibitors (eg. SAHA, Tubacin), IKK inhibitors, P38MAPK inhibitors, HSP90 inhibitors (eg 17-AAG), and akt inhibitors (eg. Perifosine).

Further, the preferred chemotherapeutic agents used in combination with the compounds of the present invention include without limitation melphalan, doxorubicin (including lyophilized), dexamethasone, prednisone, thalidomide, lenalidomide, bortezomib, and NPI0052.

In any case, the multiple chemotherapeutic agents (at least one of which is a compound of the present invention) may be administered in any order or even simultaneously. If simultaneously, the multiple chemotherapeutic agents may be provided in a single, unified form, or in multiple forms (by way of example only, either as a single pill or as two separate pills). One of the chemotherapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating HDAC-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of HDAC-mediated disorders.

All references, patents or applications, U.S. or foreign, cited in the application are hereby incorporated by reference as if written herein.

### GENERAL SYNTHETIC METHODS FOR PREPARING COMPOUNDS

Molecular embodiments of the present invention can be synthesized using standard synthetic techniques known to those of skill in the art. Compounds of the present invention can be synthesized using the general synthetic procedures set forth in Schemes I-VI. 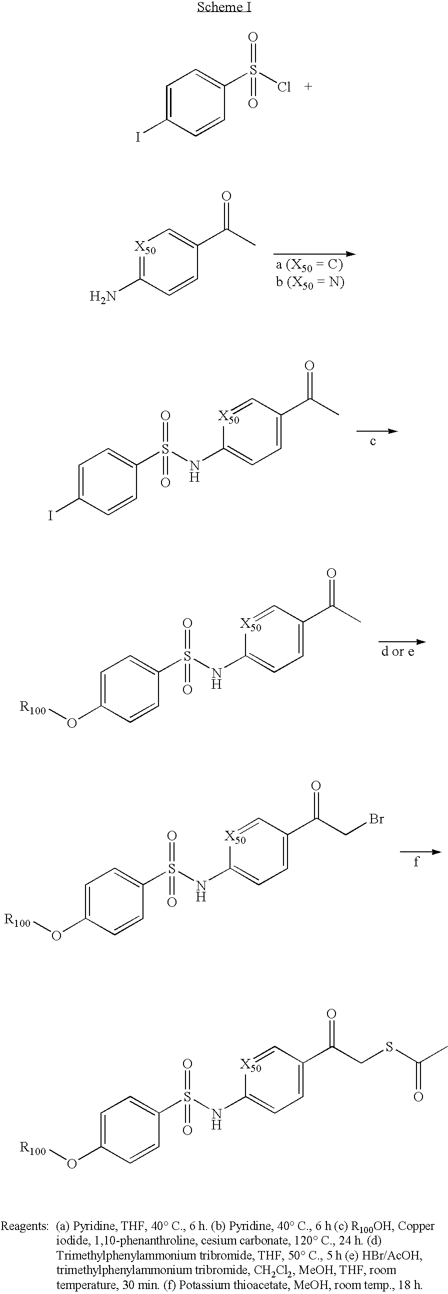 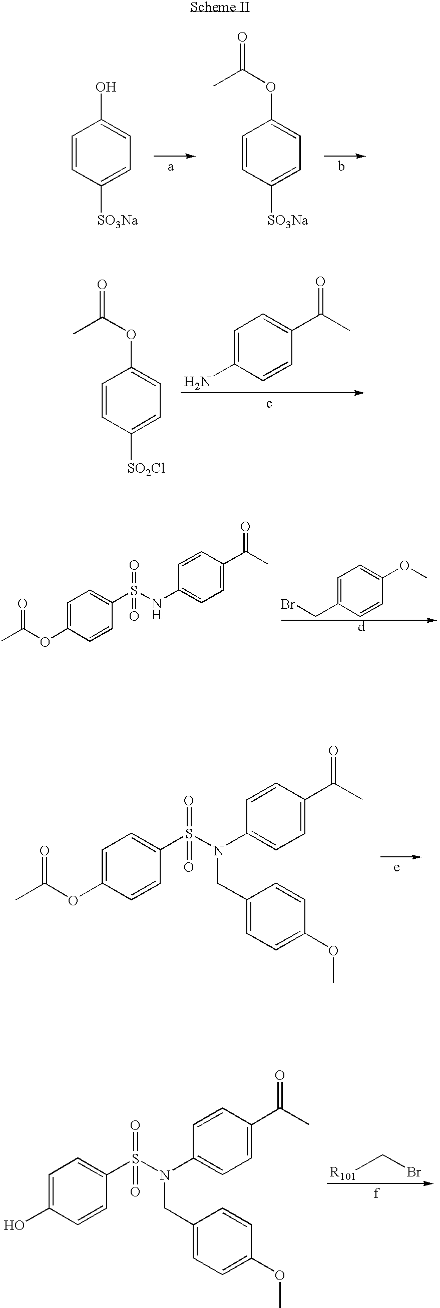 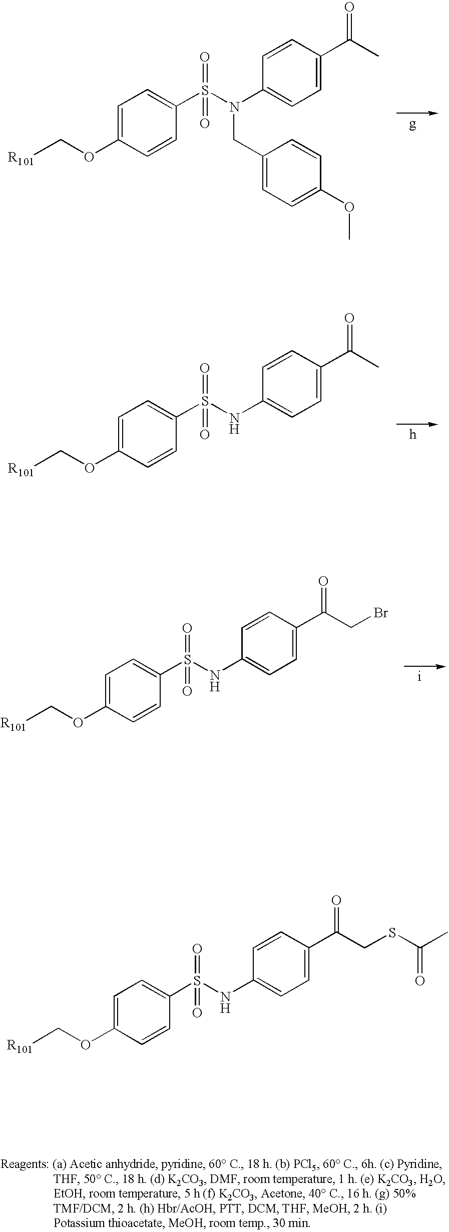 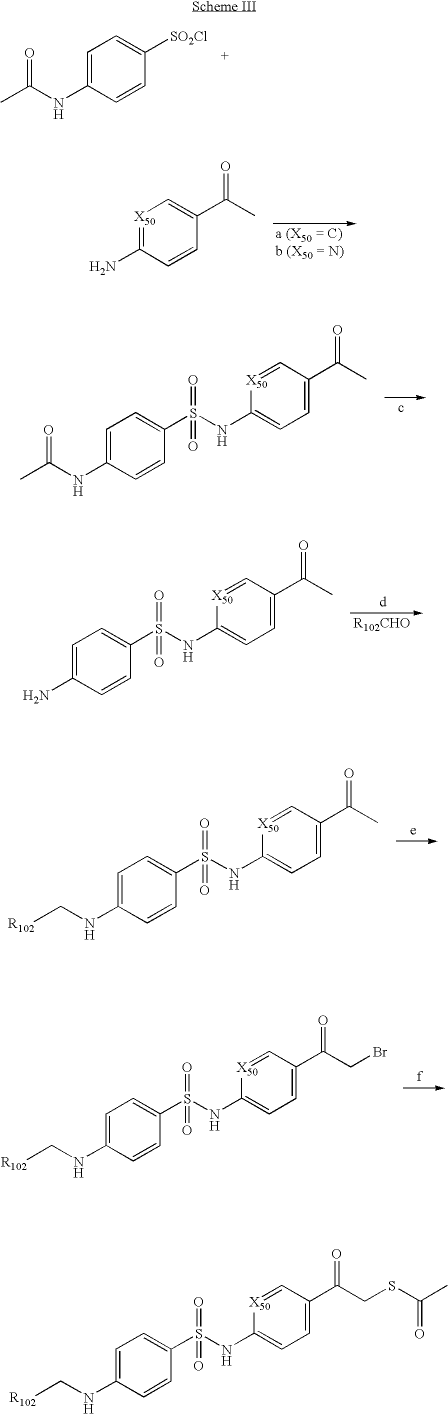 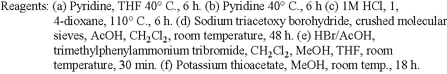 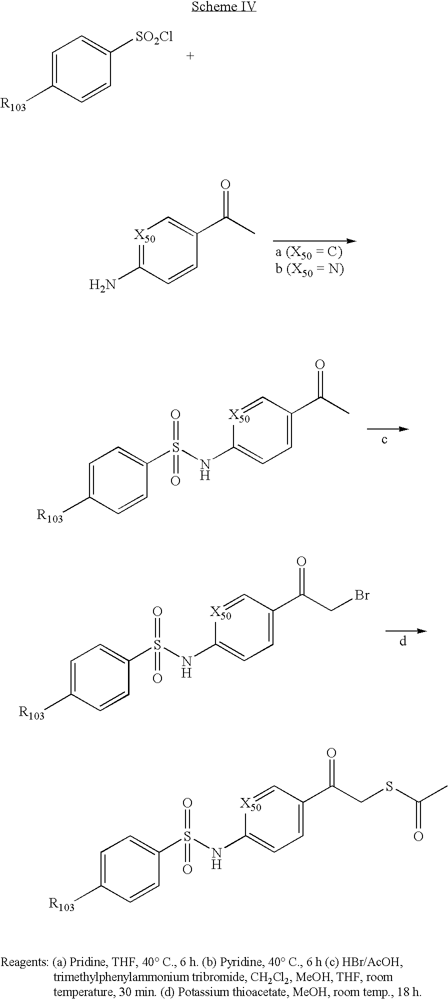 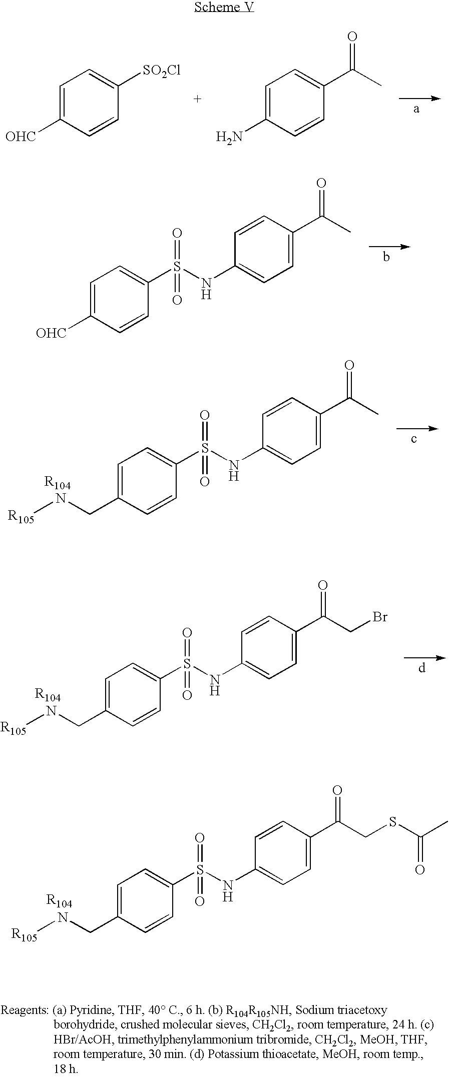
The invention is further illustrated by the following examples.

### EXAMPLE 1

### Thioacetic acid S-{2-oxo-2-[4-(4-o-tolyloxy-benzenesulfonylamino)-phenyl]-ethyl} ester

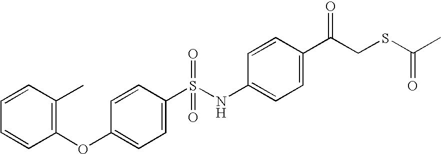 Step 1: N-(4-Acetyl-phenyl)-4-o-tolyloxy-benzenesulfonamide 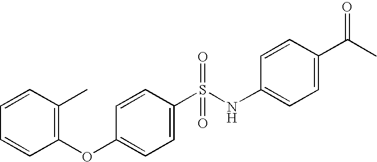
A mixture of 4-o-tolyloxy-benzenesulfonyl chloride (1 g, 3.54 mmol), 1-(4-amino-phenyl)-ethanone (0.62 g, 4.6 mmol), and pyridine (1.9 mL, 14 mmol) in THF (10 mL) was heated to 40° C. for 6 h. The solvent was removed in vacuo and the residue was partitioned between EtOAc and 1M aqueous HCl. The aqueous layer was extracted with EtOAc (3×). The organics were combined, dried over Na₂SO₄ and evaporated to dryness to afford 1.2 g of N-(4-acetyl-phenyl)-4-o-tolyloxy-benzenesulfonamide as a white solid. ¹H NMR(400 MHz, CDCl₃) δ 7.86 (d, 2H), 7.77 (d, 2H), 7.21-7.15 (m, 6H), 6.87 (d, 2H), 2.54 (s, 3H), 2.12 (s, 3H).
Step 2: N-[4-(2-Bromo-acetyl)-phenyl]-4-o-tolyloxy-benzenesulfonamide 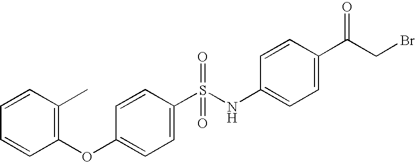
A mixture of N-(4-acetyl-phenyl)-4-o-tolyloxy-benzenesulfonamide (1.2 g, 3.12 mmol) and trimethylphenylammonium tribromide (1.3 g, 3.4 mmol) in THF (20 mL) was heated to 40° C. for 2 h. The solvent was removed in vac o and the residue was partitioned between EtOAc and water. The aqueous layer was extracted with EtOAc (3×). The organics were combined, dried over Na₂SO₄ and evaporated to dryness to afford 2 g of a mixture of N-[4-(2-bromo-acetyl)-phenyl]-4-o-tolyloxy-benzenesulfonamide, N-[4-(2,2-dibromo-acetyl)-phenyl]-4-o-tolyloxy-benzenesulfonamide and unreacted starting material.
Step 3: Thioacetic acid S-{2-oxo-2-[4-(4-o-tolyloxy-benzenesulfonylamino)-phenyl]-ethyl} ester 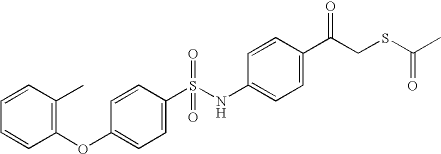
A mixture of N-[4-(2-bromo-acetyl)-phenyl]-4-o-tolyloxy-benzenesulfonamide (2 g, 4.4 mmol) and potassium thioacetate (594 mg, 5.2 mmol) in MeOH (20 mL) was stirred at room temperature for 18 h. The solvent was removed in vac o and the residue was partitioned between CH₂Cl₂ and water. The aqueous layer was extracted with CH₂Cl₂ (3×). The organics were combined, dried over Na₂SO₄ and evaporated to dryness to afford a crude compound that was purified by flash chromatography (silica gel, 1:1 EtOAc:hexane). Thioacetic acid S-{2-oxo-2-[4-(4-o-tolyloxy -benzenesulfonylamino)-phenyl]-ethyl} ester was obtained as a white solid (1.12 g). ¹H NMR (400 MHz, DMSO-d₆) δ 10.90 (s, 1H), 7.90 (d, 2H), 7.82 (d, 2H), 7.34 (d, 1H), 7.21 (m, 4H), 7.03 (d, 1H), 6.99 (d, 2H), 4.41 (s, 2H), 2.36 (s, 3H), 2.06 (s, 3H). LCMS: 456 (M+1)⁺.

### EXAMPLE 2

This example is intentionally left blank.

### EXAMPLE 3

### Thioacetic acid S-{2-oxo-2-[4-(4-phenoxy-benzenesulfonylamino)-phenyl]-ethyl} ester

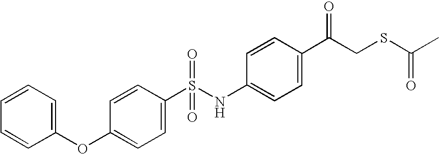 Thioacetic acid S-{2-oxo-2-[4-(4-phenoxy-benzenesulfonylamino)-phenyl]-ethyl} ester was synthesized as described in EXAMPLE 1 using 4-phenoxy-benzenesulfonyl chloride and 1-(4-amino-phenyl)-ethanone as starting materials. ¹H NMR(400 MHz, DMSO-d₆) δ 10.93 (s, 1H), 7.91 (d, 2H), 7.84 (d, 2H), 7.45 (m, 2H), 7.24 (m, 3H), 7.10 (m, 4H), 4.42 (s, 2H), 2.35 (s, 3H). LCMS: 442 (M+1)⁺.

### EXAMPLE 4

### Thioacetic acid S-(2-{4-[4-(4-chloro-phenoxy)-benzenesulfonylamino]-phenyl}-2-oxo-ethyl) ester

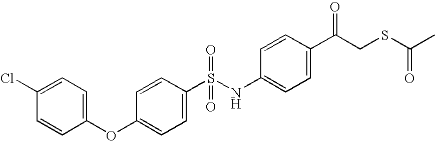 Thioacetic acid S-(2-{4-[4-(4-chloro-phenoxy)-benzenesulfonylamino]-phenyl}-2-oxo-ethyl) ester was synthesized as described in EXAMPLE 1 using 4-(4-chloro-phenoxy)-benzenesulfonyl chloride and 1-(4-amino-phenyl)-ethanone as starting materials. ¹H NMR (400 MHz, DMSO-d₆) δ 10.93 (s, 1H), 7.90 (d, 2H), 7.84 (d, 2H), 7.49 (d, 2H), 7.23 (d, 2H), 7.16 (d, 2H), 7.11 (d, 2H), 4.41 (s, 2H), 2.36 (s, 3H).

### EXAMPLE 5

### Thioacetic acid S-(2-{4-[4-(morpholine-4-sulfonyl)-benzenesulfonylamino]-phenyl}-2-oxo-ethyl) ester

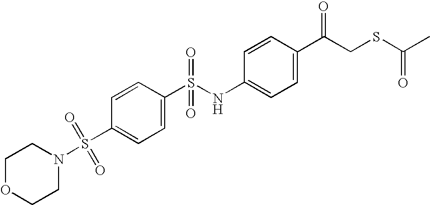 Thioacetic acid S-(2-{4-[4-(morpholine-4-sulfonyl)-benzenesulfonylamino]-phenyl}-2-oxo-ethyl) ester was synthesized as described in EXAMPLE 1 using 4-(morpholine-4-sulfonyl)-benzenesulfonyl chloride and 1-(4-amino-phenyl)-ethanone as starting materials. ¹H-NMR (400 MHz, CDCl₃) δ 8.00(d, 2H), 7.92(d, 2H), 7.82(d, 2H), 7.20(d, 2H), 4.30(s, 2H), 3.72(t, 4H), 3.00(t, 4H), 2.41(s, 3H). LCMS: 499 (M+1)⁺.

### EXAMPLE 6

This example is intentionally left blank.

### EXAMPLE 7

### Thioacetic acid S-{2-[4-(4-morpholin-4-ylmethyl-benzenesulfonylamino)-phenyl]-2-oxo-ethyl} ester

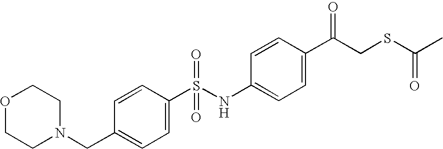 Step 1: N-(4-Acetyl-phenyl)-4-formyl-benzenesulfonamide 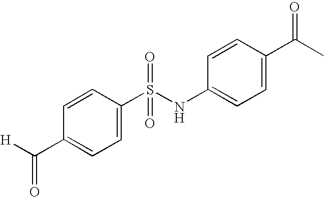
N-(4-Acetyl-phenyl)-4-formyl-benzenesulfonamide was synthesized as described in EXAMPLE 1,
Step 1 using 4-formyl-benzenesulfonyl chloride and 1-(4-amino-phenyl)-ethanone as starting materials. ¹H NMR (400 MHz, DMSO-d₆) δ 11.07 (s, 1H), 10.03 (s, 1H), 8.07 (d, 2H), 8.04 (d, 2H), 7.84 (d, 2H), 7.22 (d, 2H), 2.46 (s, 3H).
Step 2: N-(4-Acetyl-phenyl)-4-morpholin-4-ylmethyl-benzenesulfonamide 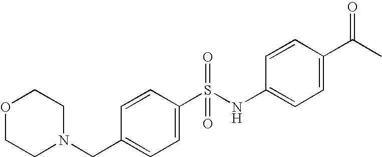
A mixture of N-(4-acetyl-phenyl)-4-formyl-benzenesulfonamide (300 mg, 0.99 mmol), morpholine (0.11 mL, 1.28 mmol), sodium triacetoxyborohydride (629 mg, 2.97 mmol), and crushed molecular sieves in CH₂Cl₂ (15 mL) was stirred at room temperature for 18 h. Water was added to the reaction mixture and it was filtered through a celite pad and the pad was washed with CH₂Cl₂. The filtrate was poured into a separatory funnel containing a saturated aqueous solution of sodium bicarbonate. The aqueous layer was extracted with CH₂Cl₂ (4×), the organics were combined, dried over Na₂SO₄ and evaporated to dryness to afford a crude that was purified by flash chromatography (silica gel, 1:1 EtOAc:hexane to 100% EtOAc). N-(4-Acetyl-phenyl)-4-morpholin-4-ylmethyl-benzenesulfonamide (326 mg) was obtained as an orange foam. ¹H NMR (400 MHz, CDCl₃) δ 7.84 (d, 2H), 7.80 (d, 2H), 7.44 (d, 2H), 7.16 (d, 2H), 3.69 (t, 4H), 3.50 (s, 2H), 2.40 (t, 4H). LCMS: 375 (M+1)⁺.
Step 3: N-[4-(2-Bromo-acetyl)-phenyl]-4-morpholin-4-ylmethyl-benzenesulfonamide 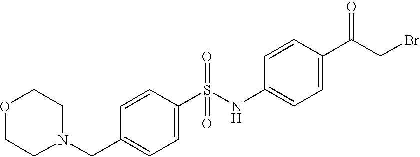
N-[4-(2-Bromo-acetyl)-phenyl]-4-morpholin-4-ylmethyl-benzenesulfonamide was synthesized as described in EXAMPLE 1, Step 2 using N-(4-acetyl-phenyl)-4-morpholin-4-ylmethyl-benzenesulfonamide as a starting material
Step 4: Thioacetic acid S-{2-[4-(4-morpholin-4-ylmethyl-benzenesulfonylamino)-phenyl]-2-oxo-ethyl} ester 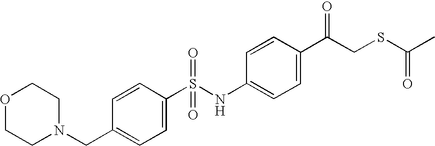
Thioacetic acid S-{2-[4-(4-morpholin-4-ylmethyl-benzenesulfonylamino)-phenyl]-2-oxo-ethyl} ester was synthesized as described in EXAMPLE 1, Step 3 using N-[4-(2-bromo-acetyl)-phenyl]-4-morpholin-4-ylmethyl-benzenesulfonamide as starting material. LCMS: 449 (M+1)⁺.

### EXAMPLE 8

### Thioacetic acid S-[2-oxo-2-(4-{4-[(pyridin-2-ylmethyl)-amino]-benzenesulfonyl amino}-phenyl)-ethyl] ester

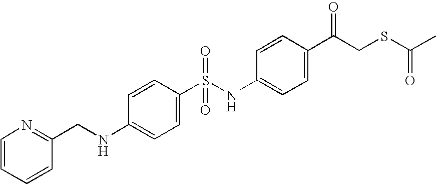 Step 1: N-(4-{[(4-acetylphenyl)amino] sulfonyl}phenyl)acetamide 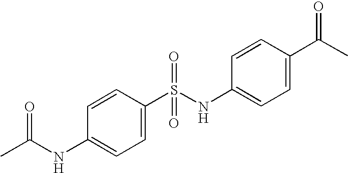
N-(4-{[(4-acetylphenyl)amino]sulfonyl}phenyl)acetamide was synthesized as described in EXAMPLE 1 using 4-acetylamino-benzenesulfonyl chloride and 1-(4-amino-phenyl)-ethanone as starting materials.
Step 2: N-(4-Acetyl-phenyl)-4-amino-benzenesulfonamide 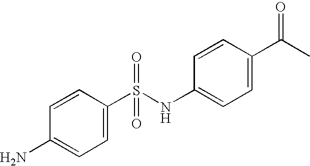
N-[4-(4-Acetyl-phenylsulfamoyl)-phenyl]-acetamide (2 g, 6.02 mmol) was dissolved in 1,4-dioxane (14 mL) and aqueous HCl (1M, 4 mL) and the resulting mixture was heated to 105° C. for 3 h. The reaction mixture was then cooled to room temperature and slowly poured into water containing Na₂CO₃. The aqueous layer was extracted with CH₂Cl₂ (3×). The organics were combined, dried over Na₂SO₄ and evaporated to dryness to afford 1.5 g of N-(4-acetyl-phenyl)-4-amino-benzenesulfonamide as a yellow solid. ¹H NMR (400 MHz, DMSO-d₆) δ 10.47 (s, 1H), 7.80 (d, 2H), 7.47 (d, 2H), 7.17 (d, 2H), 6.55 (d, 2H), 6.03 (s, 2H), 2.36 (s, 3H). LCMS: 291 (M+1)⁺.
Step 3: N-(4-Acetyl-phenyl)-4-[(pyridin-2-ylmethyl)-amino]-benzenesulfonamide 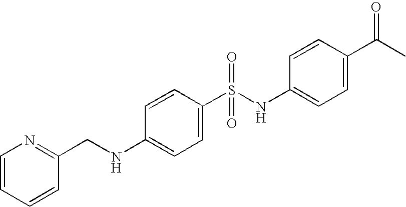
A mixture of N-(4-acetyl-phenyl)-4-amino-benzenesulfonamide (1 g, 3.44 mmol), pyridine-2-carbaldehyde (0.5 mL, 5.17 mmol), acetic acid (0.4 mL, 6.9 mmol), sodium triacetoxyborohydride (2.2 g, 10.34 mmol), and crushed molecular sieves in CH₂Cl₂ (30 mL) was stirred at room temperature for 48 h. Water was added to the reaction mixture and it was filtered through a celite pad and the pad was washed with CH₂Cl₂. The filtrate was poured into a separatory funnel containing a saturated aqueous solution of sodium bicarbonate. The aqueous layer was extracted with CH₂Cl₂ (4×), the organics were combined, dried over Na₂SO₄ and evaporated to dryness to afford a crude that was purified by preparative HPLC (8 min, 15 to 80% ACN:Water). N-(4-acetyl -phenyl)-4-[(pyridin-2-ylmethyl)-amino]-benzenesulfonamide (713 mg) was obtained as a bis-TFA salt. LCMS: 382 (M+1)⁺.
Step 4: N-[4-(2-Bromo-acetyl)-phenyl]-4-[(pyridin-2-ylmethyl)-amino]-benzene-sulfonamide 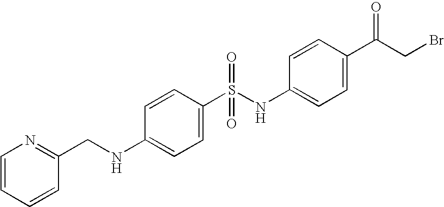
N-(4-Acetyl-phenyl)-4-[pyridin-2-ylmethyl)-amino]-benzenesulfonamide(bis-TFA salt, 713 mg, 1.17 mmol) was dissolved in CH₂Cl₂:MeOH:THF (10:1:1, 12 mL) and HBr/AcOH (33%, 2 mL). Trimethylphenylammonium tribromide (484 mg, 1.29 mmol) was added and the resulting orange mixture was stirred at room temperature for 3 h. The reaction mixture was slowly poured into a saturated aqueous solution of sodium bicarbonate. The aqueous layer was extracted with CH₂Cl₂ (4×), the organics were combined, dried over Na₂SO₄ and evaporated to dryness to afford 587 mg of a mixture of N-[4-(2-bromo-acetyl)-phenyl]-4-[(pyridin-2-ylmethyl)-amino]-benzenesulfonamide, N-[4-(2,2-dibromo-acetyl)-phenyl]-4-[(pyridin-2-ylmethyl)-amino]-benzenesulfonamide and unreacted starting material.
Step 5: Thioacetic acid S-[2-oxo-2-(4-{4-[(pyridin-2-ylmethyl)-amino]-benzene-sulfonyl amino}-phenyl)-ethyl] ester 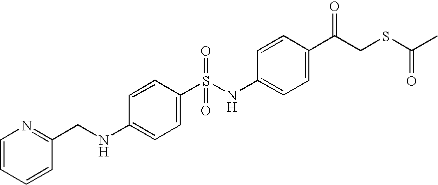
Thioacetic acid S-[2-oxo-2-(4-{4-[(pyridin-2-ylmethyl)-amino]-benzenesulfonyl amino}-phenyl)-ethyl] ester was synthesized as described in EXAMPLE 1, Step 3 using N-[4-(2-bromo-acetyl) -phenyl]-4-[(pyridin-2-ylmethyl)-amino]-benzenesulfonamide as starting material. ¹H NMR (400 MHz, CD₃OD) δ 8.45 (d, 1H), 7.80 (d, 2H), 7.68 (t, 1H), 7.55 (d, 2H), 7.32 (d, 1H), 7.25 (t, 1H), 7.17 (d, 2H), 6.56 (d, 2H), 4.42 (s, 2H), 4.31 (s, 2H), 2.33 (s, 3H). LCMS: 456 (M+1)⁺.

### EXAMPLE 9

This example is intentionally left blank.

### EXAMPLE 10

### Thioacetic acid S-(2-{4-[4-(1-methyl-piperidin-4-yloxy)-benzenesulfonylamino]-phenyl}-2-oxo-ethyl) ester

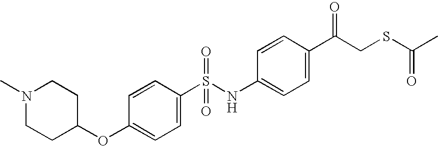 Step 1: N-(4-Acetyl-phenyl)-4-iodo-benzenesulfonamide 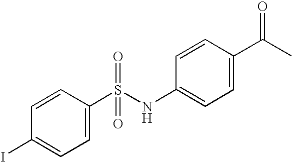
N-(4-Acetyl-phenyl)-4-iodo-benzenesulfonamide was synthesized as described in EXAMPLE 1, Step 1 using 4-iodo-benzenesulfonyl chloride and 1-(4-amino-phenyl)-ethanone as starting materials. ¹H NMR (400 MHz, DMSO-d₆) δ 10.93 (s, 2H), 7.96 (d, 2H), 7.84 (d, 2H), 7.57 (d, 2H), 7.20 (d, 2H), 2.46 (s, 3H).
Step 2: N-(4-Acetyl-phenyl)-4-(1-methyl-piperidin-4-yloxy)-benzenesulfonamide 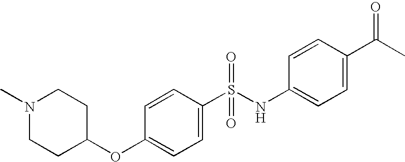
A mixture of N-(4-acetyl-phenyl)-4-iodo-benzenesulfonamide (500 mg, 1.25 mmol), 1-methyl-piperidin-4-ol (1.5 mL, 12.5 mmol), copper iodide (35 mg, 0.18 mmol), 1,10-phenanthroline (67 mg, 0.37 mmol), and cesium carbonate (1.1 g, 3.12 mmol) was heated to 120° C. for 24 h. The black reaction mixture was cooled to room temperature and partitioned between CH₂Cl₂ and a saturated aqueous ammonium chloride solution. The aqueous layer was extracted with CH₂Cl₂ (4×), the organics were combined, dried over Na₂SO₄ and evaporated to dryness to afford a crude that was purified by flash chromatography (silica gel, CH₂Cl₂ to 20% MeOH:CH₂Cl₂). N-(4-Acetyl-phenyl) -4-(1-methyl-piperidin-4-yloxy)-benzenesulfonamide (232 mg) was obtained as yellow oil. ¹H NMR (400 MHz, DMSO-d₆) δ 7.78 (m, 4H), 7.20 (d, 2H), 6.85 (d, 2H), 4.38 (m, 1H), 2.71 (m, 2H), 2.49 (s, 3H), 2.46 (m, 2H), 2.35 (s, 3H), 2.03 (m, 2H), 1.84 (m, 2H). LCMS: 389 (M+1)⁺.
Step 3: N-[4-(2-Bromo-acetyl)-phenyl]-4-(1-methyl-piperidin-4-yloxy)-benzene-sulfonamide 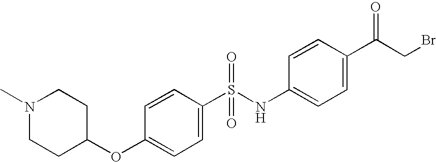
N-[4-(2-Bromo-acetyl)-phenyl]-4-(1-methyl-piperidin-4-yloxy)-benzenesulfonamide was synthesized as described in EXAMPLE 8, Step 4 using N-(4-acetyl-phenyl)-4-(1-methyl-piperidin-4-yloxy)-benzenesulfonamide as starting material.
Step 4: Thioacetic acid S-(2-{4-[4-(1-methyl-piperidin-4-yloxy)-benzenesulfony-lamino]-phenyl}-2-oxo-ethyl) ester 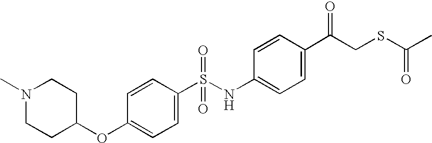
Thioacetic acid S-(2-{4-[4-(1-methyl-piperidin-4-yloxy)-benzenesulfonylamino]-phenyl}-2-oxo-ethyl) ester was synthesized as described in EXAMPLE 1, Step 3 using N-[4-(2-bromo-acetyl) -phenyl]-4-(1-methyl-piperidin-4-yloxy)-benzenesulfonamide as starting material. ¹H NMR (400 MHz, CD₃OD) δ 7.87 (d, 2H), 7.80 (d, 2H), 7.21 (d, 2H), 7.06 (d, 2H), 4.68 (m, 1H), 3.31 (s, 2H), 3.15 (m, 2H), 2.98 (m, 2H), 2.67 (s, 3H), 2.33 (s, 3H), 2.11 (m, 2H), 1.95 (m, 2H). LCMS: 463 (M+1)⁺.

### EXAMPLE 11

### Thioacetic acid S-(2-{6-[4-(3-morpholin-4-yl-propoxy)-benzenesulfonylamino]-pyridin-3-yl}-2-oxo-ethyl) ester

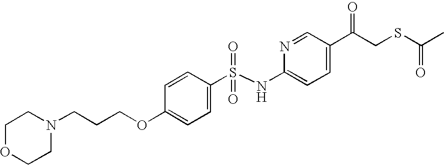 Step 1: N-(5-Acetyl-pyridin-2-yl)-4-iodo-benzenesulfonamide 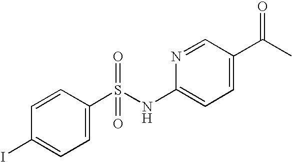
N-(5-Acetyl-pyridin-2-yl)-4-iodo-benzenesulfonamide was synthesized as described in EXAMPLE 10, Step 1 using 4-iodo-benzenesulfonyl chloride and 1-(6-amino-pyridin-3-yl)-ethanone as starting materials. ¹H NMR (400 MHz, DMSO-d₆) δ 8.59 (s, 1H), 8.13 (dd, 1H), 7.92 (d, 2H), 7.64 (d, 2H), 7.20 (d, 1H), 2.45 (s, 3H).
Step 2: N-(5-Acetyl-pyridin-2-yl)-4-(3-morpholin-4-yl-propoxy)-benzenesulfonamide 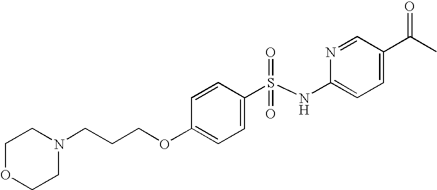
N-(5-Acetyl-pyridin-2-yl)-4-(3-morpholin-4-yl-propoxy)-benzenesulfonamide was synthesized as described in EXAMPLE 10, Step 2 using 3-morpholin-4-yl-propan-1-ol and N-(5-acetyl-pyridin-2-yl)-4-iodo-benzenesulfonamide as starting materials. ¹H NMR (400 MHz, DMSO-d₆) δ 8.65 (s, 1H), 8.09 (d, 1H), 7.83 (d, 2H), 7.11 (d, 1H), 7.05 (d, 2H), 4.06 (t, 2H), 3.55 (m, 4H), 2.50-2.32 (m, 9H), 1.87 (m, 2H). LCMS: 420 (M+1)⁺.
Step 3: N-[5-(2-Bromo-acetyl)-pyridin-2-yl]-4-(3-morpholin-4-yl-propoxy)-benzenesulfonamide 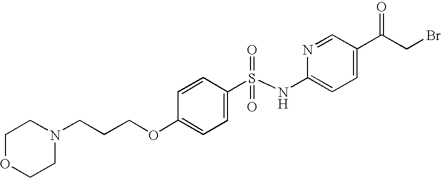
N-[5-(2-Bromo-acetyl)-pyridin-2-yl]-4-(3-morpholin-4-yl-propoxy)-benzenesulfonamide was synthesizes as described in EXAMPLE 8, Step 4 using N-(5-acetyl-pyridin-2-yl)-4-(3-morpholin-4-yl-propoxy)-benzenesulfonamide as starting material.
Step 4: Thioacetic acid S-(2-{6-[4-(3-morpholin-4-yl-propoxy)-benzenesulfonylamino]-pyridin-3-yl}-2-oxo-ethyl) ester 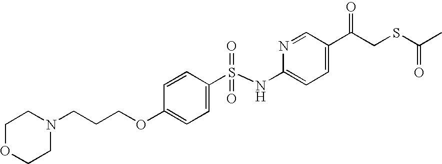
Thioacetic acid S-(2-{6-[4-(3-morpholin-4-yl-propoxy)-benzenesulfonylamino]-pyridin-3-yl}-2-oxo-ethyl) ester was synthesized as described in EXAMPLE 1, Step 3 using N-[5-(2-bromo-acetyl) -pyridin-2-yl]-4-(3-morpholin-4-yl-propoxy)-benzenesulfonamide as starting material. ¹H NMR (400 MHz, CD₃OD) δ 8.68 (s, 1H), 8.08 (d, 1H), 7.87 (d, 2H), 7.06 (d, 1H), 6.98 (d, 2H), 4.28 (s, 2H), 4.09 (m, 2H), 3.68 (t, 4H), 2.57 (m, 2H), 2.50 (m, 4H), 2.34 (s, 3H). LCMS: 494 (M+1)⁺.

### EXAMPLE 12

### Thioacetic acid S-(2-{4-[4-(3-morpholin-4-yl-propoxy)-benzenesulfonylamino]-phenyl}-2-oxo-ethyl) ester

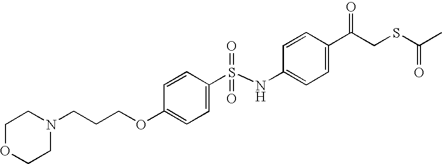 Step 1: Sodium 4-acetoxy-benzenesulfonate 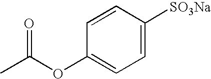
Acetic anhydride (250 mL) and pyridine (16 g, 202.28 mmol) were added to sodium 4-hydroxybenzenesulfonate (36 g, 183.52 mmol) and the resulting solution was allowed to react, with stirring, overnight while the temperature was maintained at 60-70° C. until done by TLC. The mixture was concentrated by evaporation under vacuum using a rotary evaporator. This resulted in 35 g (80%) of sodium 4-acetoxybenzenesulfonate as a dark red solid.
Step 2: Acetic acid 4-chlorosulfonyl-phenyl ester 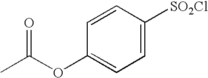
Phosphorus pentachloride (61 g, 292.93 mmol) was added to sodium 4-acetoxybenzenesulfonate (35 g, 146.94 mmol) and the resulting solution was allowed to react, with stirring, for 6 h while the temperature was maintained at 60° C. The reaction mixture was then quenched by the adding 300 ml of H₂O/ice. The resulting solution was extracted (2×) with 300 ml of CH₂Cl₂ and the organic layers combined, dried over MgSO₄, and concentrated by evaporation under vacuum using a rotary evaporator. This resulted in 35 g (61%) of acetic acid 4-chlorosulfonyl-phenyl ester as yellow oil.
Step 3: Acetic acid 4-(4-acetyl-phenylsulfamoyl)-phenyl ester 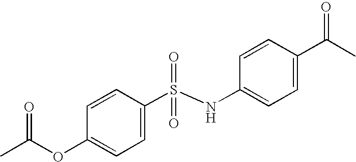
Acetic acid 4-(4-acetyl-phenylsulfamoyl)-phenyl ester was synthesized as described in EXAMPLE 1, Step 1 using acetic acid 4-chlorosulfonyl-phenyl ester and 1-(4-amino-phenyl)-ethanone as starting materials.
Step 4: Acetic acid 4-[(4-acetyl-phenyl)-(4-methoxy-benzyl)-sulfamoyl]-phenyl ester 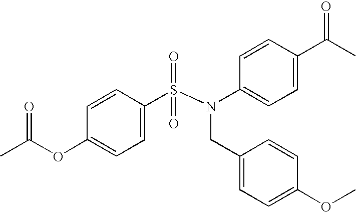
To a mixture of 4-(4-acetyl-phenylsulfamoyl)-phenyl ester (7.73 g, 23.19 mmol) and K₂CO₃ (6.41 g, 46.38 mmol) in DMF (150 ml) was added 1-(bromomethyl)-4-methoxybenzene (4.66 g, 23.18 mmol). The resulting solution was allowed to react, with stirring, for 1 hour while the temperature was maintained at room temperature until the reaction was done by TLC. The reaction mixture was then quenched by the adding 300 ml of H₂O. The resulting solution was extracted (3×) with 300 ml of EtOAc and the organic layers were combined and dried over Na₂SO₄. The filtrate was concentrated by evaporation under vacuum using a rotary evaporator. This resulted in 10.3 g (98%) of acetic acid 4-[(4-acetyl-phenyl)-(4-methoxy-benzyl)-sulfamoyl]-phenyl ester as a white solid.
Step 5: N-(4-Acetyl-phenyl)-4-hydroxy-N-(4-methoxy-benzyl)-benzenesulfonamide 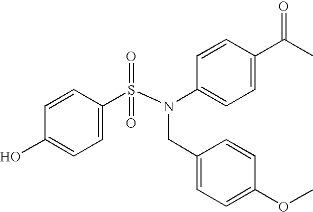
To a mixture of acetic acid 4-[(4-acetyl-phenyl)-(4-methoxy-benzyl)-sulfamoyl]-phenyl ester (10.3 g, 22.71 mmol), K₂CO₃ (3.71 g) in ethanol (100 ml) was added H₂O (50 ml) and the resulting solution was allowed to react, with stirring, for 20 minutes while the temperature was maintained at room temperature until done by TLC. The mixture was concentrated by evaporation under vacuum using a rotary evaporator. The resulting solution was extracted (3×) with 100 ml of EtOAc and the organic layers combined and dried over Na₂SO₄. The residue was purified by flash chromatography (Silica gel, eluting with a 1:1 EtOAc/Hexanes). The final product was purified by recrystallization from EtOAc/Hexanes in the ratio 1:1. This resulted in 4.1 g (44%) of N-(4-acetyl-phenyl)-4-hydroxy-N-(4-methoxy-benzyl)-benzenesulfonamide as a white solid. ¹H-NMR (400 MHz, CDCl₃) δ 7.78 (d, 2H), 7.52 (d, 2H), 7.09 (d, 4H), 6.88 (d, 2H), 6.71 (d, 2H), 4.68 (s, 2H), 3.72 (s, 3H), 2.53 (s, 3H).
Step 6: N-(4-Acetyl-phenyl)-N-(4-methoxy-benzyl)-4-(3-morpholin-4-yl-propoxy)-benzenesulfonamide 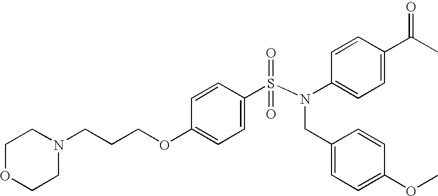
1.0 g (2.43 mmol) of N-(4-acetyl-phenyl)-4-hydroxy-N-(4-methoxy-benzyl)-benzenesulfonamide was stirred in 5 mL of dry acetone in a 20 mL septa sealed vial equipped with stir bar and N₂ balloon. 671 mg (4.86 mmol) of potassium carbonate was then added and stirred for 2 minutes at room temperature. 607 mg (2.91 mmol) of 4-(3-bromo-propyl)-morpholine was then added in one portion. The reaction was heated to 40° C. and stirred for 16 hours. The reaction was then cooled to room temperature and poured into 50 mL of H₂O. The aqueous phase was extracted 3× Ethyl Acetate. The organics were combined, washed 2×H₂O, dried over anhydrous Sodium sulfate, filtered and concentrated to give 1.1 g (84% yield) of N-(4-acetyl-phenyl)-N-(4-methoxy-benzyl)-4-(3-morpholin-4-yl-propoxy)-benzenesulfonamide as an off white solid. ¹H-NMR (400 MHz, CDCl₃) δ 7.79(d, 2H), 7.53(d, 2H), 7.09(d, 2H), 7.07(d, 2H), 6.92(d, 2H), 6.70(d, 2H), 4.64(s, 2H), 4.07(t, 2H), 3.72(t, 4H), 3.70(s, 3H), 2.54(t, 4H), 2.51(t, 2H), 2.49(s, 3H), 2.01(tt, 2H). LCMS: 540 (M+1)⁺.
Step 7: N-(4-Acetyl-phenyl)-4-(3-morpholin-4-yl-propoxy)-benzenesulfonamide 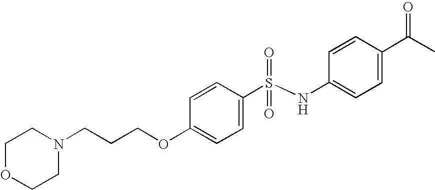
500 mg (0.93 mmol) of N-(4-acetyl-phenyl)-N-(4-methoxy-benzyl)-4-(3-morpholin-4-yl-propoxy)-benzenesulfonamide was dissolved in 3 mL of Dichloromethane in a 20 mL septa sealed vial equipped with stir bar. 3 mL of Trifluoroacetic acid was slowly added and stirred at room temperature for 2 hours upon which time the reaction was a dark pink/purple color. All volatiles were removed via N₂ stream with mild heating. The material was dissolved in a small amount of Ethyl Acetate and loaded directly onto a 5 mL pre-packed SDE silica column that was equilibrated with 50% Ethyl Acetate/Hexanes. Ten column volumes of 50% Ethyl Acetate/Hexanes were passed through removing impurities, then three column volumes of 100% Ethyl Acetate. Elution of product was obtained with 95%Dichloromethane/Methanol. Product fractions were combined and concentrated to give 338 mg (87% yield) of pure N-(4-acetyl-phenyl)-4-(3-morpholin-4-yl-propoxy)-benzenesulfonamide as a light brown oil. ¹H-NMR (400 MHz, CDCl₃) δ 7.82(d, 2H), 7.61(d, 2H), 7.02(d, 2H), 6.62(d, 2H), 3.90(t, 2H), 3.72(tt, 4H), 2.59(s, 3H), 2.38(tt, 4H), 2.34(t, 2H), 1.91(t, 2H). LCMS: 419 (M+1)⁺.
Step 8: N-[4-(2-Bromo-acetyl)-phenyl]-4-(3-morpholin-4-yl-propoxy)-benzenesulfonamide 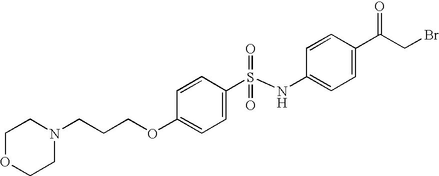
N-[4-(2-Bromo-acetyl)-phenyl]-4-(3-morpholin-4-yl-propoxy)-benzenesulfonamide was synthesized as described in EXAMPLE 8, Step 4 using N-(4-acetyl-phenyl)-4-(3-morpholin-4-yl-propoxy)-benzenesulfonamide as starting material.
Step 9: Thioacetic acid S-(2-{4-[4-(3-morpholin-4-yl-propoxy)-benzenesulfonylamino]-phenyl}-2-oxo-ethyl) ester 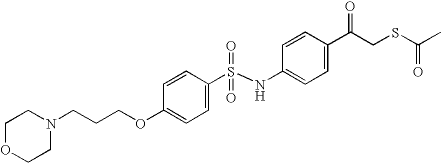
Thioacetic acid S-(2-{4-[4-(3-morpholin-4-yl-propoxy)-benzenesulfonylamino]-phenyl}-2-oxo-ethyl) ester was synthesized as described in EXAMPLE 1, Step 3 using N-[4-(2-bromo-acetyl) -phenyl]-4-(3-morpholin-4-yl-propoxy)-benzenesulfonamide as starting material. ¹H-NMR (400 MHz, CDCl₃) δ 7.88(d, 2H), 7.78(d, 2H), 7.19(d, 2H), 6.82(d, 2H), 4.30(s, 2H), 4.24(t, 2H), 4.10(t, 2H), 4.04(t, 2H), 3.62(t, 2H), 3.26(t, 2H), 2.82(tt, 2H), 2.46(t, 2H), 2.40(s, 3H). LCMS: 493 (M+1)⁺.

### EXAMPLE 13

Thioacetic acid S-(2-oxo-2-{4-[4-(4-phenyl-butoxy)-benzenesulfonylamino]-phenyl}-ethyl) ester 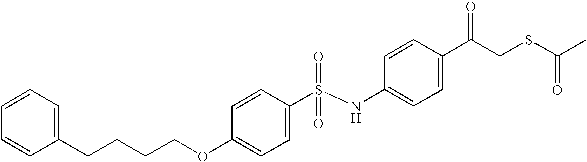
Thioacetic acid S-(2-oxo-2-{4-[4-(4-phenyl-butoxy)-benzenesulfonylamino]-phenyl}-ethyl) ester was synthesized as described in EXAMPLE 10 using 4-phenyl-butan-1-ol and N-(4-acetyl-phenyl)-4-iodo-benzenesulfonamide as starting materials. ¹H NMR (400 MHz, DMSO-d₆) δ 10.81 (s, 1H), 7.88 (d, 2H), 7.75 (d, 2H), 7.22 (m, 7H), 7.06 (d, 2H), 4.40 (s, 2H), 4.02 (t, 2H), 2.61 (m, 2H), 2.35 (s, 3H), 1.68 (m, 4H). LCMS: 498 (M+1)⁺.

### EXAMPLE 14

### Thioacetic acid S-(2-oxo-2-{6-[4-(4-phenyl-butoxy)-benzenesulfonylamino]-pyridin-3-yl}-ethyl) ester

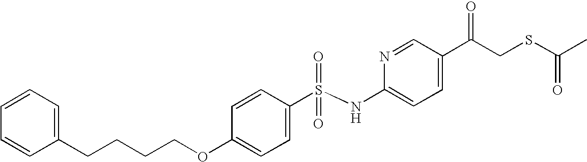 Thioacetic acid S-(2-oxo-2-{6-[4-(4-phenyl-butoxy)-benzenesulfonylamino]-pyridin-3-yl}-ethyl) ester was synthesized as described in EXAMPLE 11 using 4-phenyl-butan-1-ol and N-(5-acetyl -pyridin-2-yl)-4-iodo-benzenesulfonamide as starting materials. ¹H NMR (400 MHz, DMSO-d₆) δ 8.75 (s, 1H), 8.16 (d, 1H), 7.85 (d, 2H), 7.28-7.15 (m, 6H), 7.06 (d, 2H), 4.41 (s, 2H), 4.04 (m, 2H), 2.62 (m, 2H), 2.35 (m, 2H), 1.70 (m, 4H). LCMS: 499 (M+1)⁺.

### EXAMPLE 15

### Thioacetic acid S-(2-oxo-2-{4-[4-(pyridin-2-ylmethoxy)-benzenesulfonylamino]-phenyl}-ethyl) ester

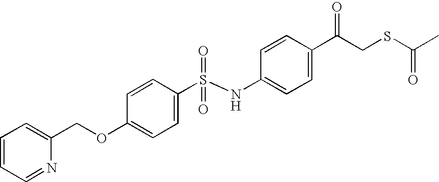 Thioacetic acid S-(2-oxo-2-{4-[4-(pyridin-2-ylmethoxy)-benzenesulfonylamino]-phenyl}-ethyl) ester was synthesized as described in EXAMPLE 10 using N-(4-acetyl-phenyl)-4-iodo-benzenesulfonamide and 2-pyridylcarbinol as starting materials. ¹H NMR (400 MHz, DMSO-d₆) δ 10.82 (s, 1H), 8.55 (d, 2H), 7.74-7.90 (m, 5H), 7.48 (d, 1H), 7.33 (dd, 1H), 7.21 (d, 2H), 7.18 (d, 2H), 5.21 (s, 2H), 4.38 (s, 2H), 2.32 (s, 3H) LCMS: 455.6 (M−1)⁻.

### EXAMPLE 16

### Thioacetic acid S-(2-oxo-2-{4-[4-(3-pyridin-3-yl-propoxy)-benzenesulfonylamino]-phenyl}-ethyl) ester

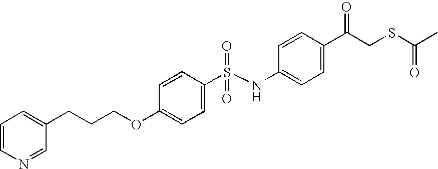 Thioacetic acid S-(2-oxo-2-{4-[4-(3-pyridin-2-yl-propoxy)-benzenesulfonylamino]-phenyl-ethyl) ester was synthesized as described in EXAMPLE 10 using N-(4-acetyl-phenyl)-4-iodo-benzenesulfonamide and 3-pyridinepropanol as starting materials. ¹H NMR (400 MHz, DMSO-d₆) δ 10.85 (s, 1H), 8.60 (d, 1H), 7.84 (d, 2H), 7.74 (d, 2H), 7.65 (m, 1H), 7.15-7.26 (m, 4H), 7.02 (d, 2H), 4.39 (s, 2H), 4.03 (t, 2H), 2.84 (t, 2H), 2.33 (s, 3H), 2.09 (m, 2H). LCMS: 483.6 (M−1)⁻.

### EXAMPLE 17

### Thioacetic acid S-(2-oxo-2-{4-[4-(3-pyridin-2-yl-propoxy)-benzenesulfonylamino]-phenyl}-ethyl) ester

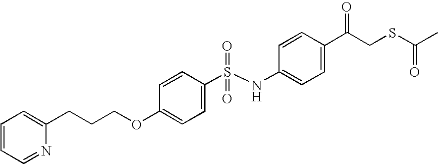 Thioacetic acid S-(2-oxo-2-{4-[4-(3-pyridin-2-yl-propoxy)-benzenesulfonylamino]-phenyl}-ethyl) ester was synthesized as described in EXAMPLE 10 using N-(4-acetyl-phenyl)-4-iodo-benzenesulfonamide and 2-pyridinepropanol as starting materials. ¹H NMR (400 MHz, DMSO-d₆) δ 10.80 (s, 1H), 8.45 (d, 1H), 7.84 (d, 2H), 7.74 (d, 2H), 7.66 (t, 1H), 7.15-7.26 (m, 4H), 7.02 (d, 2H), 4.39 (s, 2H), 4.03 (t, 2H), 2.84 (t, 2H), 2.33 (s, 3H), 2.09 (m, 2H). LCMS: 485.4 (M+1)⁺.

### EXAMPLE 18

### Thioacetic acid S-(2-oxo-2-{6-[4-(2-pyridin-2-yl-ethoxy)-benzenesulfonylamino]-pyridin-3-yl}-ethyl) ester

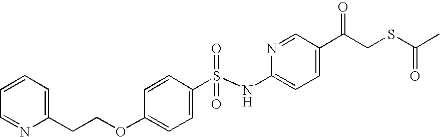 Thioacetic acid S-(2-oxo-2-6-[4-(2-pyridin-2-yl-ethoxy)-benzenesulfonylamino]-pyridin-3-yl}-ethyl) ester was synthesized as described in EXAMPLE 11 using N-(5-acetyl-pyridin-2-yl)-4-iodo-benzenesulfonamide and 2-(2-hydroxyethyl)-pyridine as starting materials. ¹H NMR (400 MHz, CD₃OD and CDCl₃) δ 8.62 (m, 2H), 8.04 (dd, 1H), 7.95 (t, 1H), 7.82 (d, 2H), 7.52 (d, 1H), 7.45 (m, 1H), 7.13 (d, 1H), 6.87 (d, 2H), 4.35 (t, 2H), 4.16 (s, 2H), 2.32 (s, 3H). LCMS: 472.0 (M+1)⁺.

### EXAMPLE 19

### Thioacetic acid S-(2-oxo-2-{4-[4-(2-piperidin-1-yl-ethoxy)-benzenesulfonylamino]-phenyl}-ethyl) ester

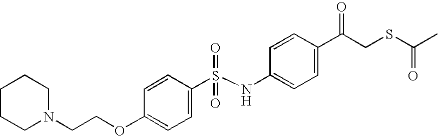 Thioacetic acid S-(2-oxo-2-{4-[4-(2-piperidin-1-yl-ethoxy)-benzenesulfonylamino]-phenyl}-ethyl) ester was synthesized as described in EXAMPLE 10 using N-(4-acetyl-phenyl)-4-iodo-benzenesulfonamide and 2-piperidin-1-yl-ethanol as starting materials. ¹H NMR (400 MHz, CD₃OD) δ 7.88 (d, 2H), 7.82 (d, 2H), 7.21 (d, 2H), 7.08 (d, 2H), 4.35 (m, 4H), 3.31 (m, 2H), 3.15 (m, 4H), 2.35 (s, 3H), 1.82 (m, 4H), 1.63 (m, 2H). LCMS: 477 (M+1)⁺.

### EXAMPLE 20

### Thioacetic acid S-(2-{4-[4-(2-morpholin-4-yl-ethoxy)-benzenesulfonylamino]-phenyl}-2-oxo-ethyl) ester

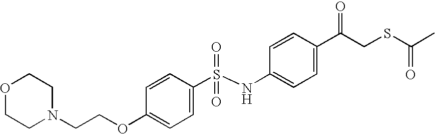 Thioacetic acid S-(2-{4-[4-(2-morpholin-4-yl-ethoxy)-benzenesulfonylamino]-phenyl}-2-oxo-ethyl) ester was synthesized as described in EXAMPLE 10 using N-(4-acetyl-phenyl)-4-iodo-benzenesulfonamide and 2-morpholin-4-yl-ethanol as starting materials. ¹H NMR (400 MHz, DMSO-d₆) δ 7.85 (d, 2H), 7.78 (d, 2H), 7.20 (d, 2H), 7.00 (d, 2H), 4.33 (s, 2H), 4.14 (t, 2H), 3.66 (t, 4H), 2.77 (t, 2H), 2.54 (t, 4H), 2.33 (s, 3H). LCMS: 479 (M+1)⁺.

### EXAMPLE 21

### Thioacetic acid S-(2-oxo-2-{4-[4-(2-pyridin-2-yl-ethoxy)-benzenesulfonylamino]-phenyl}-ethyl) ester

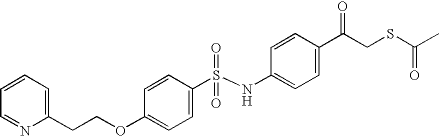 Thioacetic acid S-(2-oxo-2-{4-[4-(2-pyridin-2-yl-ethoxy)-benzenesulfonylamino]-phenyl}-ethyl) ester was synthesized as described in EXAMPLE 10 using N-(4-acetyl-phenyl)-4-iodo-benzenesulfonamide and 2-(2-hydroxyethyl)-pyridine as starting materials. ¹H NMR (400 MHz, CD₃OD) δ 8.70 (d, 1H), 8.41 (t, 1H), 7.95 (d, 1H), 7.74-7.90 (m, 5H), 7.20 (d, 2H), 7.01 (d, 2H), 4.43 (t, 2H), 4.34 (s, 2H), 3.45 (t, 2H), 2.35 (s, 3H). LCMS: 469.6 (M−1)⁻.

### EXAMPLE 22

### Thioacetic acid S-(2-oxo-2-{4-[4-(3-piperidin-1-yl-propoxy)-benzenesulfonylamino]-phenyl}-ethyl) ester

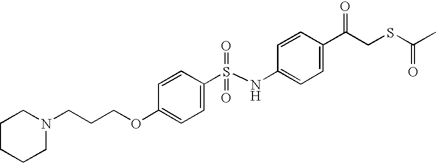 Thioacetic acid S-(2-oxo-2-{4-[4-(3-piperidin-1-yl-propoxy)-benzenesulfonylamino]-phenyl}-ethyl) ester was synthesized as described in EXAMPLE 10 using N-(4-acetyl-phenyl)-4-iodo-benzenesulfonamide and 3-piperidin-1-yl-propan-1-ol as starting materials. ¹H NMR (400 MHz, CD₃OD) δ 7.88 (d, 2H), 7.81 (d, 2H), 7.21 (d, 2H), 7.03 (d, 2H), 4.35 (s, 2H), 4.13 (t, 2H), 3.55 (m, 2H), 3.26 (m, 2H), 2.93 (t, 2H), 2.35 (s, 3H), 2.20 (m, 2H), 1.96 (m, 2H), 1.83 (m, 1H), 1.72 (m, 2H), 1.52 (m, 1H). LCMS: 491 (M+1)⁺.

### EXAMPLE 23

### Thioacetic acid S-(2-oxo-2-{6-[4-(pyridin-2-ylmethoxy)-benzenesulfonylamino]-pyridin-3-yl}-ethyl) ester

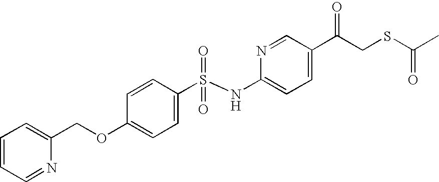 Thioacetic acid S-(2-oxo-2-{6-[4-(pyridin-2-ylmethoxy)-benzenesulfonylamino]-pyridin-3-yl}-ethyl) ester was synthesized as described in EXAMPLE 11 using N-(5-acetyl-pyridin-2-yl)-4-iodo-benzenesulfonamide and 2-pyridylcarbinol as starting materials. ¹H NMR (400 MHz, DMSO-d₆) δ 8.67 (d, 1H), 8.55 (d, 1H), 7.96 (m, 1H), 7.76-7.84 (m, 3H), 7.48 (d, 1H), 7.33 (m, 1H), 7.30 (d, 2H), 6.93 (d, 1H), 5.20 (s, 2H), 4.35 (s, 2H), 2.33 (s, 3H). LCMS: 458.4 (M+1)⁺.

### EXAMPLE 24

### Thioacetic acid S-(2-oxo-2-{6-[4-(2-piperidin-1-yl-ethoxy)-benzenesulfonylamino]-pyridin-3-yl}-ethyl) ester

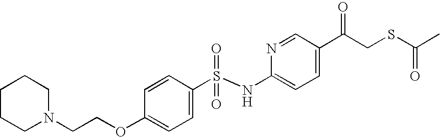 Thioacetic acid S-(2-oxo-2-{6-[4-(2-piperidin-1-yl-ethoxy)-benzenesulfonylamino]-pyridin-3-yl}-ethyl) ester was synthesized as described in EXAMPLE 11 using 2-piperidin-1-yl-ethanol and N-(5-acetyl-pyridin-2-yl)-4-iodo-benzenesulfonamide as starting materials. ¹H NMR (400 MHz, CD₃OD) δ 8.71 (s, 1H), 8.12 (d, 1H), 7.95 (d, 2H), 7.15 (d, 1H), 7.09 (d, 2H), 4.37 (m, 2H), 4.35 (s, 2H), 3.37 (m, 2H), 3.14 (m, 4H), 2.35 (s, 3H), 1.81 (m, 4H), 1.63 (m, 2H). LCMS: 478 (M+1)⁺.

### EXAMPLE 25

### Thioacetic acid S-(2-oxo-2-{6-[4-(3-piperidin-1-yl-propoxy)-benzenesulfonylamino]-pyridin-3-yl}-ethyl) ester

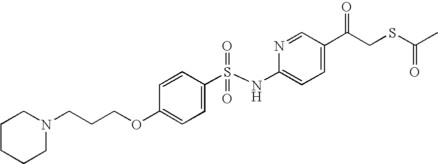 Thioacetic acid S-(2-oxo-2-{6-[4-(3-piperidin-1-yl-propoxy)-benzenesulfonylamino]-pyridin-3-yl}-ethyl) ester was synthesized as described in EXAMPLE 11 using 3-piperidin-1-yl-propan-1-ol and N-(5-acetyl-pyridin-2-yl)-4-iodo-benzenesulfonamide as starting materials. ¹H NMR (400 MHz, CD₃OD) δ 8.71 (s, 1H), 8.18 (d, 1H), 7.94 (d, 2H), 7.20 (d, 1H), 7.05 (d, 2H), 4.32 (s, 2H), 4.15 (t, 2H), 3.56 (m, 2H), 3.27 (m, 2H), 2.94 (m, 2H), 2.35 (s, 3H), 2.22 (m, 2H), 1.94 (m, 2H), 1.82 (m, 1H), 1.76 (m, 2H), 1.50 (m, 1H). LCMS: 492 (M+1)⁺.

### EXAMPLE 26

### Thioacetic acid S-(2-oxo-2-{6-[4-(2-pyrrolidin-1-yl-ethoxy)-benzenesulfonylamino]-pyridin-3-yl}-ethyl) ester

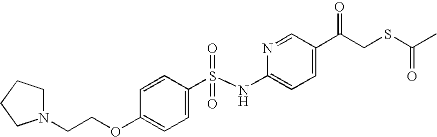 Thioacetic acid S-(2-oxo-2-{6-[4-(2-pyrrolidin-1-yl-ethoxy)-benzenesulfonylamino]-pyridin-3-yl}-ethyl) ester was synthesized as described in EXAMPLE 11 using 2-pyrrolidin-1-yl-ethanol and N-(5-acetyl-pyridin-2-yl)-4-iodo-benzenesulfonamide as starting materials. ¹H NMR (400 MHz, CD₃OD) δ 8.72 (s, 1H), 8.17 (d, 1H), 7.97 (d, 2H), 7.20 (d, 1H), 7.14 (d, 2H), 4.41 (t, 2H), 4.32 (s, 2H), 3.45 (m, 4H), 2.35 (s, 3H), 2.10 (m, 4H).

### EXAMPLE 27

### Thioacetic acid S-(2-oxo-2-{6-[4-(pyridin-3-ylmethoxy)-benzenesulfonylamino]-pyridin-3-yl}-ethyl) ester

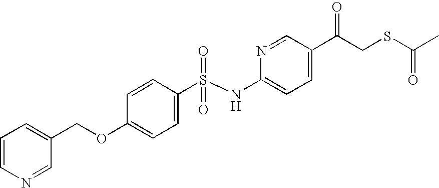 Thioacetic acid S-(2-oxo-2-{6-[4-(pyridin-3-ylmethoxy)-benzenesulfonylamino]-pyridin-3-yl}-ethyl) ester was synthesized as described in EXAMPLE 11 using N-(5-acetyl-pyridin-2-yl)-4-iodo-benzenesulfonamide and 3-pyridylcarbinol as starting materials. ¹H NMR (400 MHz, CD₃OD) δ 8.93 (s, 1H), 8.78 (d, 1H), 8.70 (s, 1H), 8.58 (d, 1H), 8.17 (dd, 1H), 7.96-8.04 (m, 3H), 7.16-7.23 (m, 3H), 5.38 (s, 2H), 4.32 (s, 2H), 2.37 (s, 3H). LCMS: 458.0 (M+1)⁺.

### EXAMPLE 28

### Thioacetic acid S-(2-oxo-2-{6-[4-(3-pyridin-2-yl-propoxy)-benzenesulfonylamino]-pyridin-3-yl}-ethyl) ester

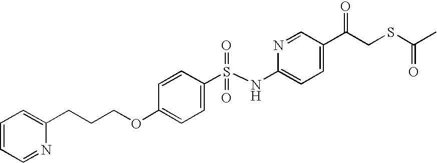 Thioacetic acid S-(2-oxo-2-{6-[4-(3-pyridin-2-yl-propoxy)-benzenesulfonylamino]-pyridin-3-yl}-ethyl) ester was synthesized as described in EXAMPLE 11 using N-(5-acetyl-pyridin-2-yl)-4-iodo-benzenesulfonamide and 2-pyridinepropanol as starting materials. ¹H NMR (400 MHz, DMSO-d₆) δ 8.73 (s, 1H), 8.46 (d, 1H), 8.13 (d, 1H), 7.84 (d, 2H), 7.66 (t, 1H), 7.25 (d, 1H), 7.10-7.20 (m, 2H), 7.02 (d, 2H), 4.39 (s, 2H), 4.05 (t, 2H), 2.85 (t, 2H), 2.33 (s, 3H), 2.10 (m, 2H). LCMS: 484.6 (M−1)⁻.

### EXAMPLE 29

### Thioacetic acid S-(2-{6-[4-(1-methyl-piperidin-4-yloxy)-benzenesulfonylamino]-pyridin-3-yl}-2-oxo-ethyl) ester

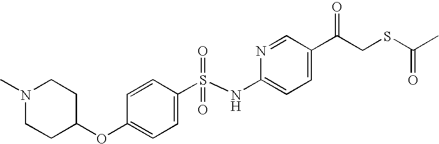 Thioacetic acid S-(2-{6-[4-(1-methyl-piperidin-4-yloxy)-benzenesulfonylamino]-pyridin-3-yl{-2-oxo-ethyl) ester was synthesized as described in EXAMPLE 11 using N-(5-acetyl-pyridin-2-yl)-4-iodo-benzenesulfonamide and 1-methyl-piperidin-4-ol as starting materials. ¹H NMR (400 MHz, CD₃OD) δ 8.72 (s, 1H), 8.18 (d, 1H), 7.95 (d, 2H), 7.21 (d, 1H), 7.13 (d, 2H), 4.32 (s, 2H), 3.61 (m, 1H), 3.40 (m, 1H), 3.18 (m, 2H), 2.95 (m, 1H), 2.90 (s, 3H), 2.40 (m, 1H), 2.35 (s, 3H), 2.25 (m, 1H), 2.08 (m, 1H), 1.85 (m, 1H). LCMS: 464 (M+1)⁺.

### EXAMPLE 30

### Thioacetic acid S-(2-{6-[4-(1-methyl-piperidin-3-yloxy)-benzenesulfonylamino]-pyridin-3-yl}-2-oxo-ethyl) ester

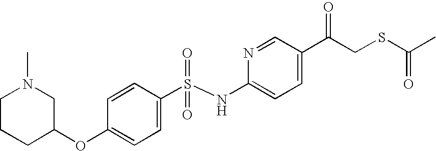 Thioacetic acid S-(2-}6-[4-(1-methyl-piperidin-3-yloxy)-benzenesulfonylamino]-pyridin-3-yl}-2-oxo-ethyl) ester was synthesized as described in EXAMPLE 11 using N-(5-acetyl-pyridin-2-yl)-4-iodo-benzenesulfonamide and l-methyl-piperidin-3-ol as starting materials. ¹H NMR (400 MHz, CDCl₃) δ 8.93 (s, 1H), 8.18 (d, 1H), 7.84 (d, 2H), 7.38 (d, 1H), 7.01 (d, 2H), 4.66 (m, 1H), 4.24 (s, 2H), 3.15 (m, 1H), 2.86 (m, 1H), 2.52-2.45 (m, 5H), 2.39 (s, 3H), 2.03-1.93 (m, 2H), 1.81 (m, 1H), 1.60 (m, 1H). LCMS: 464 (M+1)⁺.

### EXAMPLE 31

Thioacetic acid S-(2-{6-[4-(1-methyl-piperidin-3-ylmethoxy)-benzenesulfonylamino]-pyridin-3-yl}-2-oxo-ethyl) ester 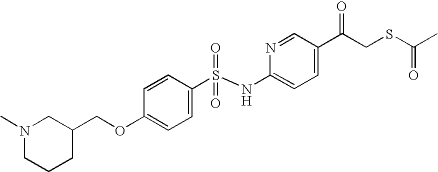
Thioacetic acid S-(2-{6-[4-(1-methyl-piperidin-3-ylmethoxy)-benzenesulfonylamino]-pyridin-3-yl}-2-oxo-ethyl) ester was synthesized as described in EXAMPLE 11 using N-(5-acetyl-pyridin-2-yl)-4-iodo-benzenesulfonamide and (1-methyl-piperidin-3-yl)-methanol as starting materials. ¹H NMR (400 MHz, DMSO-d₆) δ 9.40 (bs, 1H), 8.75 (s, 1H), 8.17 (d, 1H), 7.88 (d, 2H), 7.17 (bs, 1H), 7.10 (d, 2H), 4.42 (s, 2H), 4.05-3.88 (m, 2H), 3.53-3.41 (m, 2H), 2.78 (m, 4H), 2.36 (s, 3H), 2.21 (m, 1H), 1.91-1.64 (m, 4H), 1.24 (m, 1H). LCMS: 478 (M+1)⁺.

### EXAMPLE 32

### Thioacetic acid S-(2-{6-[4-(1-methyl-2-piperidin-1-yl-ethoxy)-benzenesulfonylamino]-pyridin-3-yl}-2-oxo-ethyl) ester

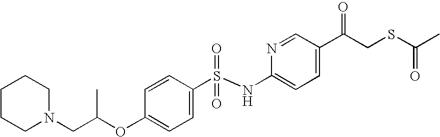 Thioacetic acid S-(2-{6-[4-(1-methyl-2-piperidin-1-yl-ethoxy)-benzenesulfonylamino]-pyridin-3-yl}-2-oxo-ethyl) ester was synthesized as described in EXAMPLE 11 using N-(5-acetyl-pyridin-2-yl)-4-iodo-benzenesulfonamide and 1-piperidin-1-yl-propan-2-ol as starting materials. ¹H NMR (400 MHz, DMSO-d₆) δ 9.49 (bs, 1H), 8.77 (s, 1H), 8.17 (d, 1H), 7.91 (d, 2H), 7.21 (d, 1H), 7.16 (d, 2H), 5.03 (m, 1H), 4.22 (s, 2H), 3.42 (m, 4H), 2.97 (m, 2H), 2.36 (s, 3H), 1.76-1.63 (m, 5H), 1.38 (m, 1H), 2.25 (d, 3H). LCMS: 492 (M+1)⁺.

### EXAMPLE 33

### Thioacetic acid S-(2-oxo-2-{6-[4-(3-pyrrolidin-1-yl-propoxy)-benzenesulfonylamino]-pyridin-3-yl}-ethyl) ester

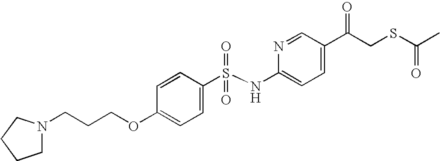 Step 1: N-(5-Acetyl-pyridin-2-yl)-4-(3-hydroxy-propoxy)-benzenesulfonamide 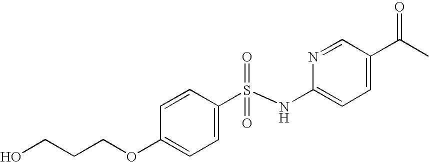
N-(5-Acetyl-pyridin-2-yl)-4-(3-hydroxy-propoxy)-benzenesulfonamide was synthesized as described in EXAMPLE 10, Step 2 using propane-1,3-diol and N-(5-acetyl-pyridin-2-yl)-4-iodo-benzenesulfonamide as starting materials. LCMS: 351 (M+1)⁺.
Step 2: N-(5-Acetyl-pyridin-2-yl)-4-(3-bromo-propoxy)-benzenesulfonamide 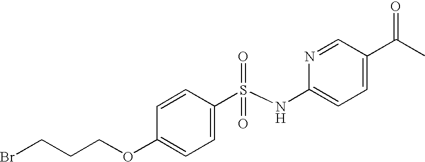
A mixture of N-(5-acetyl-pyridin-2-yl)-4-(3-hydroxy-propoxy)-benzenesulfonamide (5.4 g, 15.4 mmol) and triphenyl phosphine (4.8 g, 18.5 mmol) in dichloromethane (200 mL) was colled to 0 C. Carbon tetrabromide (6.1, 18.5 mmol) in dichloromethane (10 mL) was then added dropwise. The ice bath was removed and the resulting mixture was stirred at room temperature for 2 h. The volatiles were removed and the residue was by purified by flash chromatography (silica gel, DCM:ACN) to afford 3.3 g (51%) of N-(5-acetyl-pyridin-2-yl)-4-(3-bromo-propoxy)-benzenesulfonamide as a white solid. ¹H NMR (400 MHz, CDCl₃) δ 8.94 (s, 1H), 8.20 (d, 1H), 7.85 (d, 2H), 7.41 (d, 1H), 6.94 (d, 2H), 4.41 (t, 2H), 3.58 (t, 2H), 2.53 (s, 3H), 2.32 (m, 2H).
Step 3: N-(5-Acetyl-pyridin-2-yl)-4-(3-pyrrolidin-1-yl-propoxy)-benzenesulfonamide 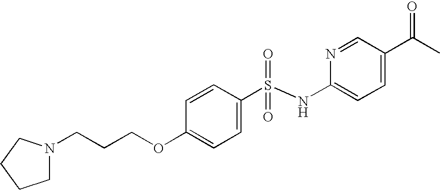
A mixture of N-(5-acetyl-pyridin-2-yl)-4-(3-bromo-propoxy)-benzenesulfonamide (0.5 g, 1.21 mmol), potassium carbonate (0.25 g, 1.81 mmol) and pyrrolidine (0.4 mL, 4.84 mmol) in Ethanol (10 mL) was heated to 70 C for 5 h. The potassium carbonate was filtered and the filtrate was evaporated to dryness. The residue was purified by flash chromatography (silica gel, DCM:MeOH) to afford N-(5-acetyl-pyridin-2-yl)-4-(3-pyrrolidin-1-yl-propoxy)-benzenesulfonamide. LCMS: 404 (M+1)⁺.
Step 4: Thioacetic acid S-(2-oxo-2-{6-[4-(3-pyrrolidin-1-yl-propoxy)-benzenesulfonylamino]-pyridin-3-yl}-ethyl) ester 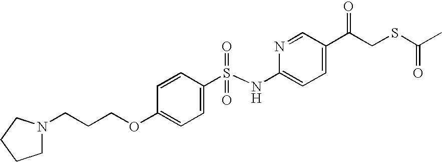
Thioacetic acid S-(2-oxo-2-{6-[4-(3-pyrrolidin-1-yl-propoxy)-benzenesulfonylamino]-pyridin-3-yl}-ethyl) ester was synthesized as described in EXAMPLE 8, Step 4 and 4 using N-(5-acetyl -pyridin-2-yl)-4-(3-pyrrolidin-1-yl-propoxy)-benzenesulfonamide as starting material. ¹H NMR (400 MHz, DMSO-d₆) δ 9.67 (bs, 1H), 8.75 (s, 1H), 8.17 (d, 1H), 7.89 (d, 2H), 7.18 (d, 1H), 7.09 (d, 2H), 4.42 (s, 2H), 4.12 (t, 2H), 3.56 (m, 2H), 3.27 (m, 2H), 3.04 (m, 2H), 2.36 (s, 3H), 2.12-1.85 (m, 6H). LCMS: 478 (M+1)⁺.
The following compounds can generally be made using the methods described above. It is expected that these compounds when made will have activity similar to those that have been made in the examples above.

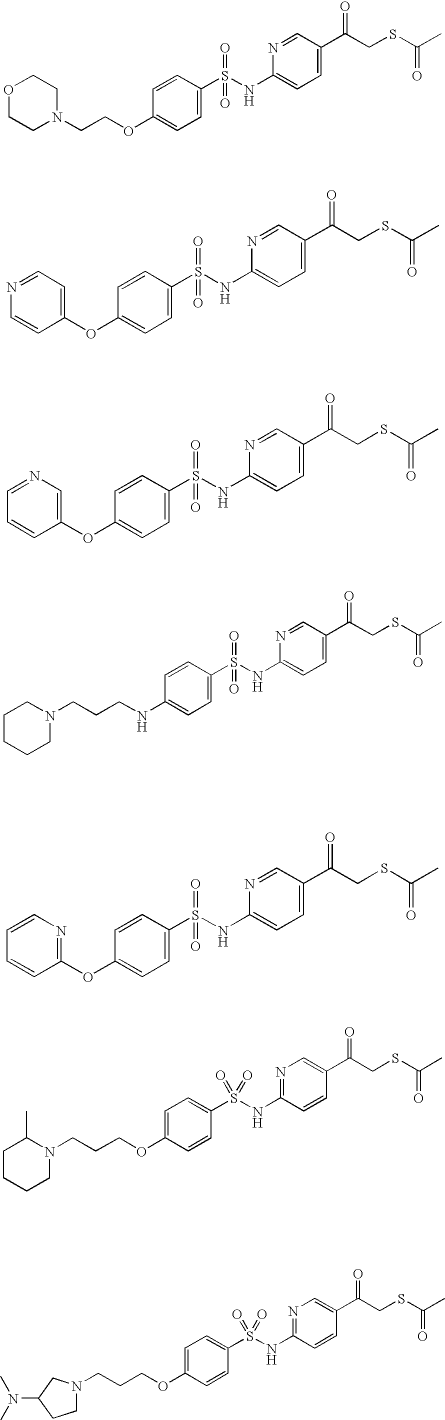

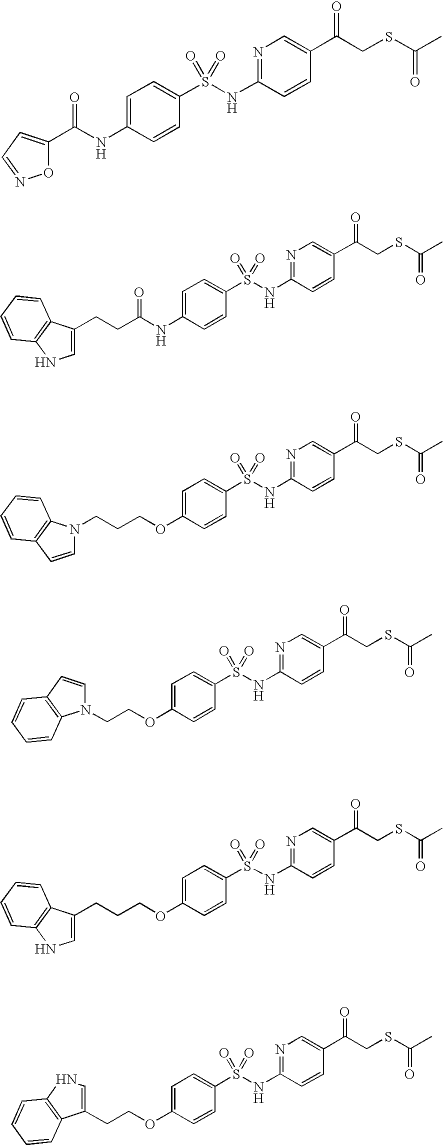

The activity of the above mentioned compounds a s HDAC inhibitors has generally been shown by the following assays. The other compounds listed above, which may not yet been made or tested, are predicted to generally have activity in these assays as well.

Inhibition Assays

1) In vitro HDAC-inhibition Assay:

This assay measures a compound's ability to inhibit acetyl-lysine deacetylation in vitro and was used as both a primary screening method as well as for IC₅₀ determinations of confirmed inhibitors. The assay is performed in vitro using an HDAC enzyme source (e.g. partially purified nuclear extract or immunopurified HDAC complexes) and a proprietary fluorescent substrate/developer system (HDAC Quantizyme Fluor de Lys Fluorescent Activity Assay, BIOMOL). The assay is run in 1,536-well Greiner white-bottom plates using the following volumes and order of addition:
Step 1: Enzyme (2.5 μL) source added to plate (from refrigerated container)
Step 2: Compounds (50 nL) added with pin transfer device
Step 3: Fluor de Lys (2.5 μL) substrate added, incubate at RT, 30 minutes
Step 4: Developer (5 μL) solution is added (containing TSA), to stop reaction
Step 5: Plate Reader—data collection

The deacetylated fluorophore is excited with 360 nm light and the emitted light (460 nm) is detected on an automated fluorometric plate reader (Aquest, Molecular Devices).

2) Cellular Histone Hyperacetylation Assays:

These two secondary assays evaluate a compound's ability to inhibit HDAC in cells by measuring cellular histone acetylation levels. The cytoblot facilitates quantitative EC₅₀ information for cellular HDAC inhibition. Transformed cell lines (e.g. HeLa, A549, MCF-7) are cultured under standard media and culture conditions prior to plating.

For Cytoblot:

Cells (approx. 2,500/well) are allowed to adhere 10-24 hours to wells of a 384-well Greiner PS assay plate in media containing 1-5% serum. Cells are treated with appropriate compound and specific concentrations for 0 to 24 hours. Cells are washed once with PBS (60 μL) and then fixed (95% ethanol, 5% acetic acid or 2% PFA) for 1 minute at RT (30 μL). Cells are blocked with 1% BSA for 1 hour and washed and stained with antibody (e.g. anti-Acetylated Histone H3, Upstate Biotechnology), followed by washing and incubation with an appropriate secondary antibody conjugated to HRP or fluorophore. For luminescence assays, signal is generated using Luminol substrate (Santa Cruz Biotechnology) and detected using an Aquest plate reader (Molecular Devices).

For Immunoblot:

Cells (4×10⁵/well) are plated into Coming 6-well dish and allowed to adhere overnight. Cells are treated with compound at appropriate concentration for 12-18 hours at 37 degrees. Cells are washed with PBS on ice. Cells are dislodged with rubber policeman and lysed in buffer containing 25 mM Tris, pH7.6; 150 mM NaCl, 25 mM MgCl₂, 1% Tween-20, and nuclei collected by centrifugation (7500 g). Nuclei are washed once in 25 mM Tris, pH7.6; 10 mM EDTA, collected by centrifugation (7500 g). Supernatant is removed and histones are extracted using 0.4 M HCl. Samples are centrifuged at 14000 g and supernatants are precipitated in 1 ml cold acetone. The histone pellet is dissolved in water and histones are separated and analyzed by SDS-PAGE Coomassie and immunoblotting (anti-acetylated histone antibodies, Upstate Biotechnology) using standard techniques.

3) Differential Cytotoxicity Assay:

HDAC inhibitors display differential cytotoxicity toward certain transformed cell lines. Cells are cultured according to standard ATCC recommended conditions that are appropriate to each cell type. Compounds were tested for their ability to kill different cell types (normal and transformed) using the ATPlite luminescence ATP detection assay system (Perkin Elmer). Assays are run in either 384-well or 1536-well Greiner PS plates. Cells (30 μL or 5 μL, respectively) are dispensed using either multichannel pipette for 384-well plates, or proprietary Kalypsys bulk liquid dispenser for 1536-well plates. Compounds added using proprietary pin-transfer device (500 nL or 5 nL) and incubated 5 to 30 hours prior to analysis. Luminescence is measured using Aquest plate reader (Molecular Devices).

The activity of some of the compounds of the invention are shown in Table 1 below. Synthesis of each compound is described in the Examples listed in the left column.

TABLE 1

| | In vitro IC₅₀ (μM) | Cellular IC₅₀ (μM) |
|---|---|---|
| | + indicates ≦1 | + indicates ≦1 |
| Example No. | − indicates >1 | − indicates >1 |
| 01 | + | + |
| 03 | + | + |
| 04 | + | + |
| 05 | + | + |
| 07 | + | + |
| 08 | + | + |
| 10 | + | + |
| 11 | + | + |
| 12 | + | + |
| 13 | + | + |
| 14 | + | + |
| 15 | + | + |
| 16 | + | − |
| 17 | + | + |
| 18 | + | + |
| 19 | + | + |
| 20 | + | + |
| 21 | + | + |
| 22 | + | + |
| 23 | + | + |
| 24 | + | + |
| 25 | + | + |
| 26 | + | + |
| 27 | + | + |
| 28 | + | + |
| 29 | + | + |
| 30 | + | + |
| 31 | + | + |
| 32 | + | + |
| 33 | + | + |

All references cited above are incorporated herein by reference in their entirety.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound having structural formula (I), 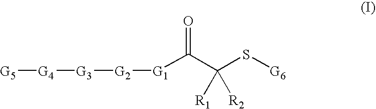
or a salt, ester, or prodrug thereof, wherein:
G₁ is selected from the group consisting of optionally substituted 5 or 6 membered aryl and optionally substituted 5 or 6 membered heteroaryl;
G₂ is selected from the group consisting of an N-sulfonamide moiety having structure (II), an S-sulfonamide moiety having structure (III), an amide of the form —NR₃C(O)—, and an amide of the form —C(O)NR₃—: 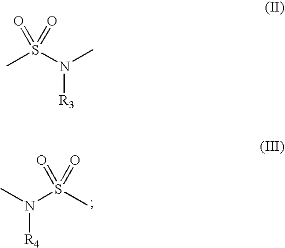
G₃ is selected from the group consisting of optionally substituted phenyl, optionally substituted 5 or 6 membered aryl, and optionally substituted 5 or 6 membered heteroaryl;
R₁ and R₂ are each independently selected from the group consisting of hydrogen, lower alkyl, halogen and perhaloalkyl, or R₁ and R₂ taken together may form an optionally substituted cycloalkyl or optionally substituted heterocycloalkyl;
R₃ and R₄ are each independently selected from the group consisting of hydrogen, optionally substituted lower alkyl, and optionally substituted aryl;
G₄ is selected from the group consisting of —(CR₅R₆)ₘ—, —(X₁)ₙ₁O(X₂)ₙ₂—, —(X₁)ₙ₁NR₇(X₂)ₙ₂—, —SO₂—, —(X₁)ₙ₁C(O)NR₇(X₂)ₙ₂—, and —(X₁)ₙ₁NR₇C(O)(X₂)ₙ₂—, wherein each may be optionally substituted with one or more R₉s attached to any carbon atom;
R₅ and R₆ are each independently selected from the group consisting of hydrogen, optionally substituted lower alkyl, optionally substituted lower alkoxy, optionally substituted aryl, and optionally substituted lower perhaloalkyl;
R₇ is selected from the group consisting of hydrogen, optionally substituted lower alkyl, optionally substituted heteroalkyl, and optionally substituted lower alkoxy;
R₉ is selected from the group consisting of lower alkyl, lower alkylene, lower alkynylene, lower alkoxy, lower amine, halogen, lower perhaloalkyl, and hydroxyl;
m is 1-6;
X₁ and X₂ are each independently selected from the group consisting of optionally substituted lower alkylene, optionally substituted alkenylene, and optionally substituted alkynylene;
n1 is 0-5;
n2 is 0-5;
G₅ is selected from the group consisting of optionally substituted aryl, optionally substituted heteroaryl, optionally substituted cycloalkyl, optionally substituted heterocycloalkyl, optionally substituted fused aryl, optionally substituted fused heteroaryl, optionally substituted fused heterocycloalkyl, and optionally substituted fused cycloalkyl; and
G₆ is selected from the group consisting of hydrogen, optionally substituted acyl, optionally substituted aryl, optionally substituted alkyl, optionally substituted heteroaryl, optionally substituted alkylthio, optionally substituted arylthio and optionally substituted heteroarylthio.

2. The compound as recited in claim 1 wherein G₂ is selected from the group consisting of N-sulfonamide or S-sulfonamide.

3. The compound as recited in claim 2 wherein G₆ is selected from the group consisting of optionally substituted acyl and hydrogen.

4. The compound as recited in claim 3 wherein G₂ is N-sulfonamide.

5. The compound as recited in claim 4 wherein G₃ is phenyl.

6. The compound as recited in claim 5 wherein G₄is —(X₁)ₙ₁O(X₂)ₙ₂— and n1 is 0.

7. The compound as recited in claim 6 wherein G₅ is selected from the group consisting of optionally substituted heterocycloalkyl, optionally substituted heteroaryl or optionally substituted aryl.

8. The compound as recited in claim 7 wherein G₅ is optionally substituted heterocycloalkyl.

9. The compound as recited in claim 7 wherein G₁ is pyridinyl.

10. The compound as recited in claim 7 wherein G₁ is phenyl.

11. The compound as recited in claim 5 wherein G₄ is —(CR₅R₆)ₘ—.

12. The compound as recited in claim 5 wherein G₄ is —(X₁)ₙ₁NR₇(X₂)ₙ₂— and n1 is 0.

13. The compound as recited in claim 1 wherein G₅ is selected from the group consisting of optionally substituted phenyl, N-morpholino, pyridinyl, optionally substituted piperidino, and pyrrolidinyl.

14. The compound as recited in claim 1 wherein said compound is selected from the group consisting of Examples 1, 3-5, 7, 8, and 10-33.

15. A pharmaceutical composition comprising a compound as recited in claim 1 together with at least one pharmaceutically acceptable carrier, diluent or excipient.

16. The compound as recited in claim 1 wherein the compound or pharmaceutically acceptable salt, ester or prodrug thereof is capable of inhibiting the catalytic activity of histone deacetylase (HDAC).

17. A method of treatment of a HDAC-related disease in a patient in need thereof comprising the administration of the following in any order:
a) a therapeutically effective amount of a compound as recited in claim 1; and
b) null or another chemotherapeutic agent.

18. The method as recited in claim 17 wherein said chemotherapeutic agent is one selected from the group consisting of aromatase inhibitors, antiestrogen, anti-androgen, or a gonadorelin agonists, topoisomerase 1 and 2 inhibitors, microtubule active agents, alkylating agents, antineoplastic antimetabolite, or platin containing compound, lipid or protein kinase targeting agents, protein or lipid phosphatase targeting agents, anti-angiogentic agents, agents that induce cell differentiation, bradykinin 1 receptor and angiotensin II antagonists, cyclooxygenase inhibitors, heparanase inhibitors, lymphokines or cytokine inhibitors, bisphosphanates, rapamycin derivatives, anti-apoptotic pathway inhibitors, apoptotic pathway agonists, PPAR agonists, inhibitors of Ras isoforms, telomerase inhibitors, protease inhibitors, metalloproteinase inhibitors, and aminopeptidase inhibitors.

19. The method as recited in claim 18 wherein said chemotherapeutic agent is useful for the treatment of multiple myeloma and is selected from the group consisting of alkylating agents, anthracyclines, corticosteroids, IMiDs, protease inhibitors, IGF-1 inhibitors, CD40 antibody, Smac mimetics, FGF3 modulator, mTOR inhibitor, HDAC inhibitors, IKK inhibitors, P38MAPK inhibitors, HSP90 inhibitor, and akt inhibitor.

20. The method as recited in claim 19 wherein said chemotherapeutic agent is selected from the group consisting of melphalan, doxorubicin, dexamethasone, prednisone, thalidomide, lenalidomide, bortezomib, and NPI0052.

21. The method as recited in claim 20, wherein said disease is selected from the group consisting of a hyperproliferative condition, a neurological disorder, a cardiovascular condition, an autoimmune disease, a dermatologic disorder, and an ophthalmologic disorder.

22. The method as recited in claim 21 wherein said hyperproliferative condition is selected from the group consisting of hematologic and nonhematologic cancers.

23. The method as recited in claim 22 wherein said hematologic cancer is selected from the group consisting of multiple myeloma, leukemias, and lymphomas.

24. The method as recited in claim 23, wherein said hematologic cancer is multiple myeloma.

25. A method of inhibition of HDAC comprising contacting HDAC with a compound as recited in claim 1.

26. The compound as recited in claim 1 for use in the manufacture of a medicament for the prevention or treatment of a disease or condition ameliorated by the modulation of histone deacetylase (HDAC).
